(19) United States
(12) Patent Application Publication
(10) Pub. No.: US 2017/0071573 A1
(43) Pub. Date: Mar. 16, 2017

(54) ULTRASOUND DIAGNOSTIC APPARATUS AND CONTROL METHOD THEREOF

(71) Applicant: Toshiba Medical Systems Corporation, Otawara-shi (JP)

(72) Inventor: Sayaka TAKAHASHI, Otawara (JP)

(73) Assignee: Toshiba Medical Systems Corporation, Otawara-shi (JP)

(21) Appl. No.: 15/342,605

(22) Filed: Nov. 3, 2016

Related U.S. Application Data
(63) Continuation of application No. PCT/JP2015/063668, filed on May 12, 2015.

(30) Foreign Application Priority Data
May 12, 2014 (JP) 2014-099051

(51) Int. Cl. A61B 8/00; A61B 8/08; A61B 8/06; A61B 8/14

(52) U.S. Cl. CPC A61B 8/467; A61B 8/461; A61B 8/54; A61B 8/4245; A61B 8/14; A61B 8/488; A61B 8/06

(57) ABSTRACT

According to an embodiment, an ultrasound diagnostic apparatus comprises an ultrasound probe used for ultrasound transmission/reception and circuitry configured to detect a probe operating state, determine whether the probe operating state matches a predetermined condition; and accept an operation information input by at least one of gesture and speech by an operator of the ultrasound probe based on a determination result obtained by the determination.

### CROSS-REFERENCE TO RELATED APPLICATIONS

This application is a Continuation Application of PCT Application No. PCT/JP2015/063668, filed May 12, 2015 and based upon and claims the benefit of priority from the Japanese Patent Application No. 2014-099051, filed May 12, 2014, the entire contents of all of which are incorporated herein by reference.

### FIELD

Embodiments described herein relate generally to an ultrasound diagnostic apparatus which requires the input of operation information and a program for the apparatus.

### BACKGROUND

An ultrasound diagnostic apparatus generally includes an operation panel including a keyboard and a trackball and a display device using a liquid crystal display or the like, and is configured to input examination parameters necessary for diagnosis and make, for example, changes to the parameters by using the operation panel and the display device. However, during an examination, an operator such as a doctor or examination technician is operating a probe, and hence cannot sometimes perform an input operation on the operation panel because both hands are occupied or even one free hand cannot reach the operation panel, depending on the position of an examination target region of an object or the posture of the operator.

Under the circumstances, there has been proposed an apparatus provided with a speech recognition function to allow the operator to input operation information by speech. For example, the conventional apparatus is configured to store predetermined control words in advance, discriminate the word input by speech whether it corresponds to any of the words stored in advance, upon collation between them, and accept the word if the corresponding word is stored.

In addition, the another conventional apparatus is configured to recognize the control command input by speech and validate the input control command if the recognized control command is confirmed as a control command corresponding to the operation setting content of each element at that point of time.

### BRIEF DESCRIPTION OF THE DRAWINGS

FIG. 1 is a perspective view showing an outer appearance of an ultrasound diagnostic apparatus according to the first embodiment.

FIG. 2 is a block diagram showing the functional arrangement of the ultrasound diagnostic apparatus according to the first embodiment.

FIG. 3 is a perspective view showing an example of the positional relationship between the apparatus and an operator, which is used to explain the operation of the first embodiment.

FIG. 4 is a flowchart showing a processing procedure and processing contents of operation support control executed by the ultrasound diagnostic apparatus shown in FIG. 2.

FIG. 5 is a view showing the first example of a display screen when a gesture/speech input acceptance mode is set during an examination period.

FIG. 6 is a view showing the second example of a display screen when the gesture/speech input acceptance mode is set during an examination period.

FIG. 7 is a block diagram showing the functional arrangement of an ultrasound diagnostic apparatus according to the second embodiment.

FIG. 8 is view showing an example of the positional relationship between the apparatus and an operator, which is used to explain the operation of the second embodiment.

FIG. 9 is view showing an example of the positional relationship between the apparatus and the operator, which is used to explain the operation of the second embodiment.

FIG. 10 is a flowchart showing a processing procedure and processing contents of operation support control executed by the ultrasound diagnostic apparatus shown in FIG. 7.

FIG. 11 is a view showing the first example of a display screen when the gesture/speech input acceptance mode is set at the time of information input setting during a non-examination period.

FIG. 12 is a view showing the second example of a display screen when the gesture/speech input acceptance mode is set at the time of information input setting during a non-examination period.

FIG. 13 is a view showing the third example of a display screen when the gesture/speech input acceptance mode is set at the time of information input setting during a non-examination period.

FIG. 14 is a block diagram showing the functional arrangement of an ultrasound diagnostic apparatus according to the third embodiment.

FIG. 15 is a perspective view showing an example of the positional relationship between the apparatus and the operator, which is used to explain the operation of the third embodiment.

FIG. 16 is a flowchart showing a processing procedure and processing contents of operation support control executed by the ultrasound diagnostic apparatus shown in FIG. 14.

FIG. 17 is a view showing an example of gesture/speech input acceptance processing based on the operation support control shown FIG. 16.

FIG. 18 is a view showing the first example of a display screen when a trackball operation is required and the gesture/speech input acceptance mode is set.

FIG. 19 is a block diagram showing the functional arrangement of an ultrasound diagnostic apparatus according to the fourth embodiment.

FIG. 20 is a view showing an example of the positional relationship between the apparatus and the operator, which is used to explain the operation of the fourth embodiment.

FIG. 21 is a flowchart showing a processing procedure and processing contents of operation support control executed by the ultrasound diagnostic apparatus shown in FIG. 19.

FIG. 22 is a block diagram showing the functional arrangement of an ultrasound diagnostic apparatus according to the fifth embodiment.

FIG. 23 is a view showing an example of the positional relationship between the apparatus and the operator, which is used to explain the operation of the fifth embodiment.

FIG. 24 is a flowchart showing a processing procedure and processing contents of operation support control executed by the ultrasound diagnostic apparatus shown in FIG. 22.

FIG. 25 is a view for explaining the body axis angle of the operator relative to the vertical direction, which is used to explain the operation of the fifth embodiment.

FIG. 26 is a view showing the first example of a display screen when the gesture/speech input acceptance mode is set during an examination period (multiple operator input acceptance).

FIG. 27 is a view showing the second example of a display screen when the gesture/speech input acceptance mode is set during an examination period (multiple operator input acceptance).

FIG. 28 is a view showing an example of the positional relationship between the apparatus and the operator, which is used to explain the first modification of the fifth embodiment.

### DETAILED DESCRIPTION

According to an embodiment, an ultrasound diagnostic apparatus comprises an ultrasound probe used for ultrasound transmission/reception and circuitry configured to detect a probe operating state, determine whether the probe operating state matches a predetermined condition, and accept an operation information input by at least one of gesture and speech by an operator of the ultrasound probe based on a determination result obtained by the determination.

An embodiment will be described below with reference to the accompanying drawings.

### First Embodiment

In the first embodiment, an ultrasound diagnostic apparatus is configured to use, as gesture/speech input acceptance conditions, conditions that the operator is operating an ultrasound probe and the distance between the operator and the ultrasound diagnostic apparatus is equal to or more than a preset distance and set the gesture/speech input acceptance mode to execute gesture/speech input acceptance processing when the conditions are satisfied.

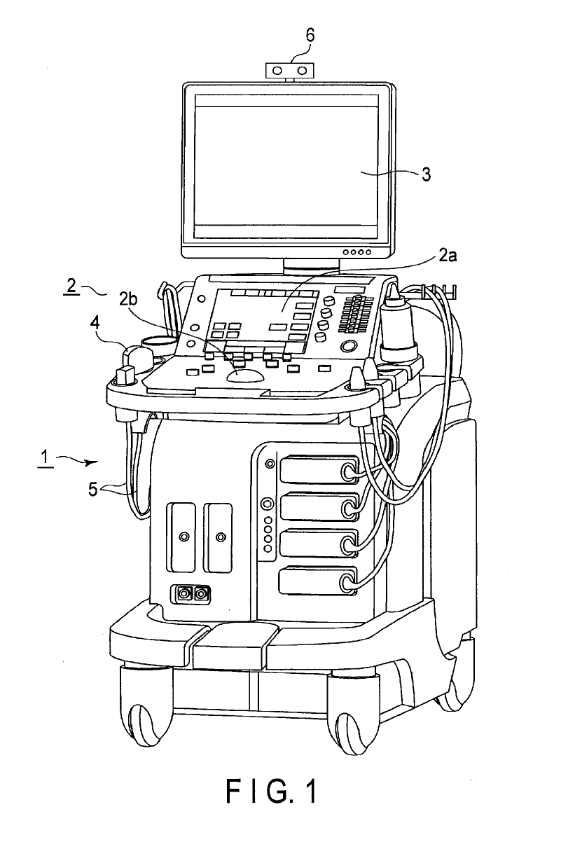

FIG. 1 is a perspective view showing an outer appearance of the ultrasound diagnostic apparatus according to the first embodiment.

The ultrasound diagnostic apparatus according to the first embodiment includes an operation panel 2 and a monitor 3 as a display device, which are arranged on the upper portion of an apparatus main body 1, and an ultrasound probe 4 housed in a side portion of the apparatus main body 1.

The operation panel 2 includes various types of switches, buttons, a trackball, a mouse, and a keyboard which are used to input, to the apparatus main body 1, various types of instructions, conditions, an instruction to set a region of interest (ROI), various types of image quality condition setting instructions, and the like from an operator. The monitor 3 is formed from, for example, a liquid crystal display, and is used to display various types of control parameters and ultrasound images during an examination. During a non-examination period, the monitor 3 is used to display various types of setting screens and the like for inputting the above setting instructions.

The ultrasound probe 4 includes N (N is an integer equal to or more than two) transducer arrays on its distal end portion. The distal end portion is brought into contact with the body surface of an object to perform ultrasound transmission/reception. Each transducer is formed from an electroacoustic conversion element, and has a function of converting an electrical driving signal into a transmission ultrasound wave at the time of transmission, and converting a reception ultrasound wave into an electrical reception signal at the time of reception. Although the first embodiment will exemplify a case in which a sector scanning ultrasound probe having a plurality of transducers is used, it is possible to use an ultrasound probe compatible for linear scanning, convex scanning, or the like.

A sensor 6 is attached to the upper portion of the housing of the display device 3. The sensor 6 is used to detect the position, direction, and movement of a person, object, or the like in a space (examination space) in which an examination is performed. The sensor 6 includes a camera 61 and a microphone 62. The camera 61 uses, for example, a CMOS (Complementary Metal Oxide Semiconductor) or CCD (Charge Coupled Device) as an imaging element, and images a person, object, or the like in an examination space, and outputs the obtained image data to the apparatus main body 1. The microphone 62 is formed from a microphone array having an array of a plurality of compact microphones. The microphone 62 detects the speech uttered by the operator in a space in which the above examination is performed, and outputs the detected speech data to the apparatus main body 1. Note that for example, a Kinect® sensor is used as the sensor 6.

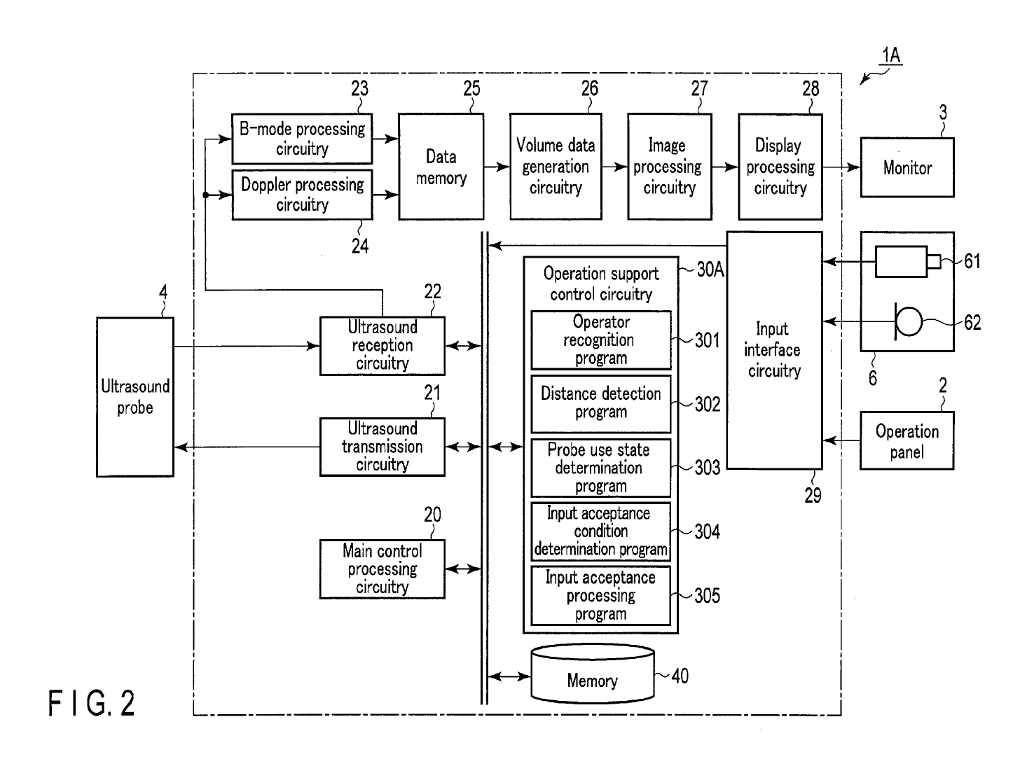

FIG. 2 is a block diagram showing the functional arrangement of the apparatus main body 1, together with its peripheral elements.

The apparatus main body 1 includes a main control processing circuitry 20, an ultrasound transmission circuitry 21, an ultrasound reception circuitry 22, an input interface circuitry 29, an operation support control circuitry 30A, and a memory 40. These circuitry are connected to each other via a bus. The main control processing circuitry 20 is constituted by, for example, a predetermined processor and a memory. The main control processing circuitry 20 comprehensively controls the overall apparatus.

The ultrasound transmission circuitry 21 includes a trigger generation circuit, delay circuit, and pulser circuit (none of which are shown). The trigger generation circuit repeatedly generates trigger pulses for the formation of transmission ultrasound waves at a predetermined rate frequency fr Hz (period: 1/fr sec). The delay circuit gives each trigger pulse a delay time necessary to focus an ultrasound wave into a beam and determine transmission directivity for each channel. The pulser circuit applies a driving pulse to the ultrasound probe 4 at the timing based on this trigger pulse.

The ultrasound reception circuitry 22 includes an amplifier circuit, A/D converter, delay circuit, and adder (none of which are shown). The amplifier circuit amplifies an echo signal received via the ultrasound probe 4 for each channel. The A/D converter converts each amplified analog echo signal into a digital echo signal. The delay circuit gives the digitally converted echo signals delay times necessary to determine reception directivities and perform reception dynamic focusing. The adder then performs addition processing for the signals. With this addition, a reflection component from a direction corresponding to the reception directivity of the echo signal is enhanced to form a composite beam for ultrasound transmission/reception in accordance with reception directivity and transmission directivity. The echo signal output from the ultrasound reception circuitry 22 is input to a B-mode processing circuitry 23 and a Doppler processing circuitry 24.

The B-mode processing circuitry 23 is constituted by, for example, a predetermined processor and a memory. The B-mode processing circuitry 23 receives an echo signal from the ultrasound reception circuitry 22, and performs logarithmic amplification, envelope detection processing, and the like for the signal to generate data whose signal intensity is expressed by a luminance level.

The Doppler processing circuitry 24 is constituted by, for example, a predetermined processor and a memory. The Doppler processing circuitry 24 extracts a blood flow signal from the echo signal received from the ultrasound reception circuitry 22, and generates blood flow data. In general, the Doppler processing circuitry 24 extracts a blood flow by CFM (Color Flow Mapping). In this case, the Doppler processing circuitry 24 analyzes the blood flow signal to obtain blood flow information such as mean velocities, variances, and powers as blood flow data at multiple points.

A data memory includes a predetermined processor. The data memory 25 generates B-mode raw data as B-mode data on three-dimensional ultrasound scanning lines by using a plurality of B-mode data received from the B-mode processing circuitry 23. The data memory 25 also generates blood flow raw data as blood flow data on three-dimensional ultrasound scanning lines by using a plurality of blood flow data received from the Doppler processing circuitry 24. Note that for the purpose of reducing noise or smooth concatenation of images, a three-dimensional filter may be inserted after the data memory 25 to perform spatial smoothing.

A volume data generation circuitry 26 is constituted by, for example, a predetermined processor and a memory. The volume data generation circuitry 26 generates B-mode volume data and blood flow volume data from the B-mode RAW data and the blood flow raw data received from the data memory 25 by executing RAW/voxel conversion.

An image processing circuitry 27 is constituted by, for example, a predetermined processor and a memory. The image processing circuitry 27 performs predetermined image processing such as volume rendering, MPR (Multi Planar Reconstruction), and MIP (Maximum Intensity Projection) for the volume data received from the volume data generation circuitry 26. Note that for the purpose of reducing noise or smooth concatenation of images, a two-dimensional filter may be inserted after the image processing circuitry 27 to perform spatial smoothing.

A display processing circuitry 28 is constituted by, for example, a predetermined processor and a memory. The display processing circuitry 28 executes various types of processing for image display associated with a dynamic range, luminance (brightness), contrast, γ curve correction, RGB conversion, and the like for various types of image data generated/processed by the image processing circuitry 27.

An input interface circuitry 29 is constituted by, for example, a predetermined processor and a memory. The input interface circuitry 29 receives the image data output from the camera 61 of the sensor 6 described above and the speech data output from the microphone 62. The received image data and speech data are saved in a buffer area in a memory 40.

An operation support control circuitry 30A is constituted by, for example, a predetermined processor and a memory. The operation support control circuitry 30A supports the input of control commands by gesture or speech of the operator during an examination, and includes, as its control functions, an operator recognition program 301, a distance detection program 302, a probe use state determination program 303, an input acceptance condition determination program 304, and an input acceptance processing program 305. Each of these control functions is implemented by causing the processor of the main control processing circuitry 20 to execute a corresponding program stored in a program memory (not shown).

The operator recognition program 301 recognizes an image of a person and the ultrasound probe 4 existing in an examination space based on image data in the examination space which is saved in the memory 40, and discriminates the person holding the ultrasound probe 4 as an operator.

The distance detection program 302 irradiates the operator with infrared light and receives reflected light by using the distance measurement light source and photoreceiver of the camera 61 of the sensor 6, and detects a distance L between an operator 7 and the monitor 3 based on the phase difference between the received reflected light and the irradiation wave or the time from the irradiation to the reception.

The probe use state determination program 303 determines whether the ultrasound probe 4 is in use, depending on whether the main control processing circuitry 20 is under the examination mode or a live ultrasound image is currently displayed on the monitor 3. Note that it is also possible to determine whether the ultrasound probe 4 is in use, depending on whether the ultrasound probe 4 is detected from the image data of the examination space based on the recognition results on the image of the person and the ultrasound probe 4 which are obtained by the operator recognition program 301.

The input acceptance condition determination program 304 determines whether the current state of the operator satisfies the gesture/speech input acceptance conditions, based on the distance detected by the distance detection program 302 and the use state of the ultrasound probe 4 which is determined by the probe use state determination program 303.

If the input acceptance condition determination program 304 determines that the current state of the operator satisfies the gesture/speech operation information input acceptance conditions, the input acceptance processing program 305 sets the gesture input acceptance mode and displays, on the display screen of the monitor 3, an icon 41 indicating that gesture/speech input is being accepted. The input acceptance processing program 305 recognizes the gesture and speech of the operator, respectively, from the image data of the operator obtained from the camera 61 of the sensor 6 and the speech data of the operator obtained by the microphone 62. The input acceptance processing program 305 then determines the validity of the operation information represented by the recognized gesture and speech, and accepts the operation information represented by the gesture and speech, if the information is valid.

### (Operation)

The input operation support operation performed by the apparatus having the above arrangement will be described next.

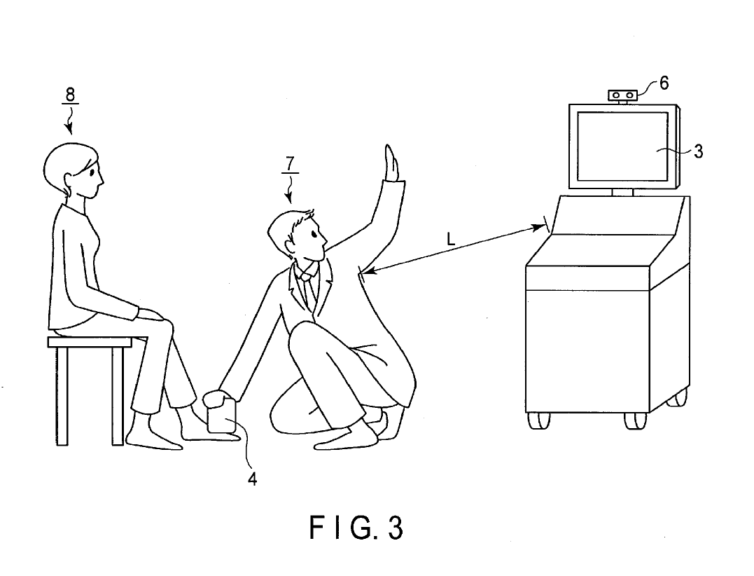

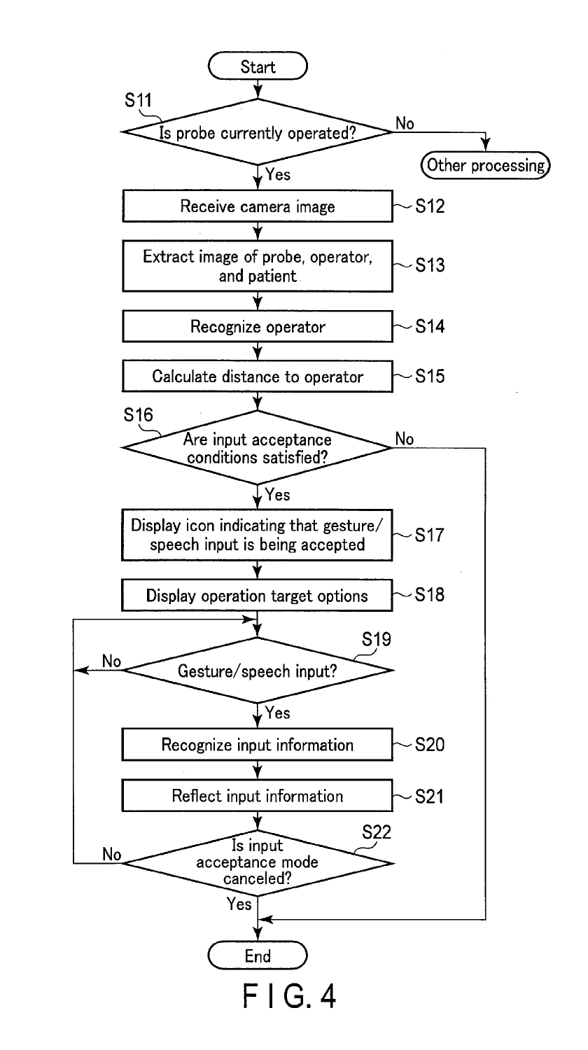

FIG. 3 is a perspective view showing an example of the positional relationship between the apparatus main body 1 and the ultrasound probe 4, the operator 7, and an object 8. FIG. 4 is a flowchart showing a processing procedure and processing contents of input operation support control executed by the operation support control circuitry 30A.

### (1) Determination of Use State of Ultrasound Probe 4

First of all, the operation support control circuitry 30A determines in step S11 whether the ultrasound probe 4 is in use under the control of the probe use state determination program 303. This determination can be made depending on whether the main control processing circuitry 20 is under the examination mode or a live ultrasound image is displayed on the monitor 3.

### (2) Recognition of Operator

The operation support control circuitry 30A executes processing for recognizing the operator in the following manner under the control of the operator recognition program 301.

First of all, in step S12, the operation support control circuitry 30A receives the image data obtained by imaging an examination space from the camera 61 of the sensor 6, and saves the data in the buffer area in the memory 40. In step S13, the operation support control circuitry 30A recognizes the image of the ultrasound probe 4 and the person from the saved image data. The recognition of the ultrasound probe 4 is performed by, for example, pattern recognition. More specifically, with respect to the saved 1-frame image data, a target region with a smaller size is set. Every time the position of this target region is shifted by one pixel, the corresponding image is collated with the image pattern of the ultrasound probe 4. If the degree of matching becomes equal to or more than a threshold, the image of the collation target is recognized as the image of the ultrasound probe 4. In step S14, the person holding the extracted ultrasound probe 4 is recognized as the operator 7.

### (3) Detection of Distance L Between Operator 7 and Monitor 3

In step S15, under the control of the distance detection program 302, the distance L between the monitor 3 and a specific region of the recognized operator 7, for example, the position of the shoulder joint on the side where the ultrasound probe 4 is not held, is detected in the following manner.

That is, the sensor 6 applies infrared light to the examination space and receives the light reflected by the operator 7 of the irradiation light by using, for example, the light source and the photoreceiver of the camera 61, which are used for distance measurement. The distance L between the sensor 6 and the position of the shoulder joint of the operator 7 on the side where he/she does not hold the ultrasound probe 4 based on the phase difference between the received reflected light and the irradiation wave or the time from the irradiation to the reception of light. Note that since the sensor 6 is integrally attached to the upper portion of the monitor 3, the distance L can be regarded as the distance between the operator and the monitor 3.

### (4) Determination on Whether Input Acceptance Conditions are Satisfied

When the calculation of the distance L is complete, it is determined whether the current state of the operator 7 satisfies the gesture/speech operation information input acceptance conditions, based on the distance L detected by the distance detection program 302 and the determination result on the use state of the ultrasound probe 4 which is determined by the probe use state determination program 303, under the control of the input acceptance condition determination program 304 in step S16. If, for example, it is determined in step S11 that the ultrasound probe 4 is in use and the distance between the operator 7 and the monitor 3 is 50 cm or more, it is determined that the input acceptance conditions are satisfied. If this determination indicates that the the input acceptance conditions are satisfied, the input operation support control is terminated without setting the gesture/speech operation information input mode.

### (5) Gesture/Speech Operation Information Input Acceptance Processing

In contrast to this, assume that it is determined in step S16 that the input acceptance conditions are satisfied. In this case, gesture/speech input acceptance processing is executed in the following manner under the control of the input acceptance processing program 305.

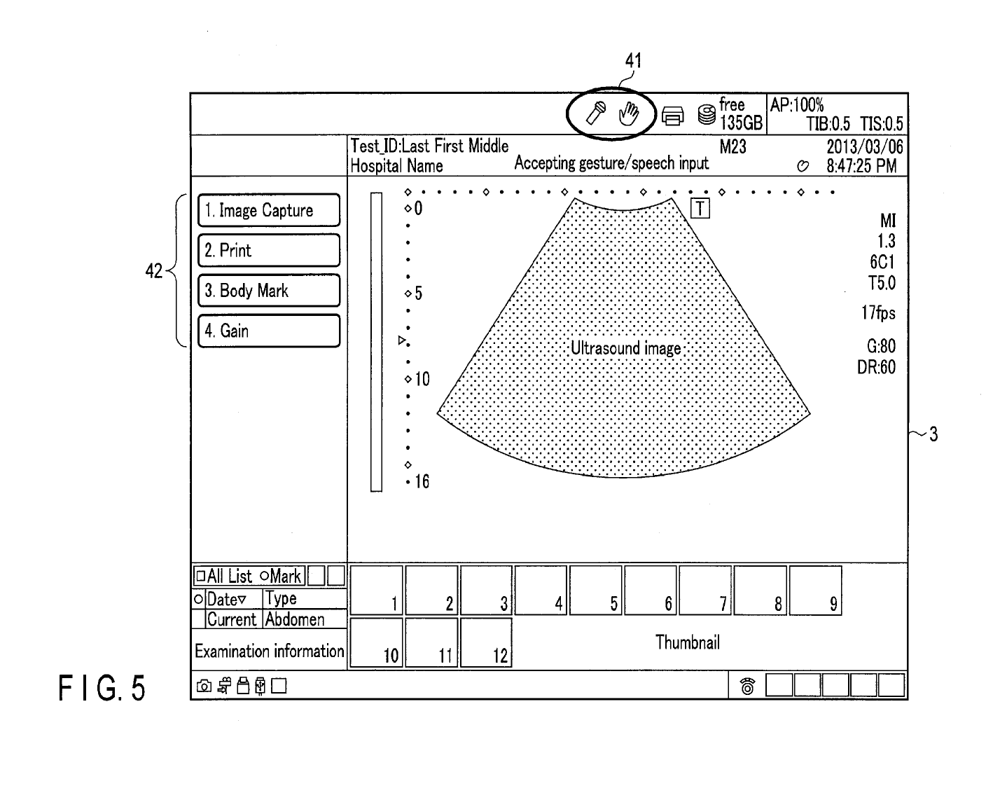

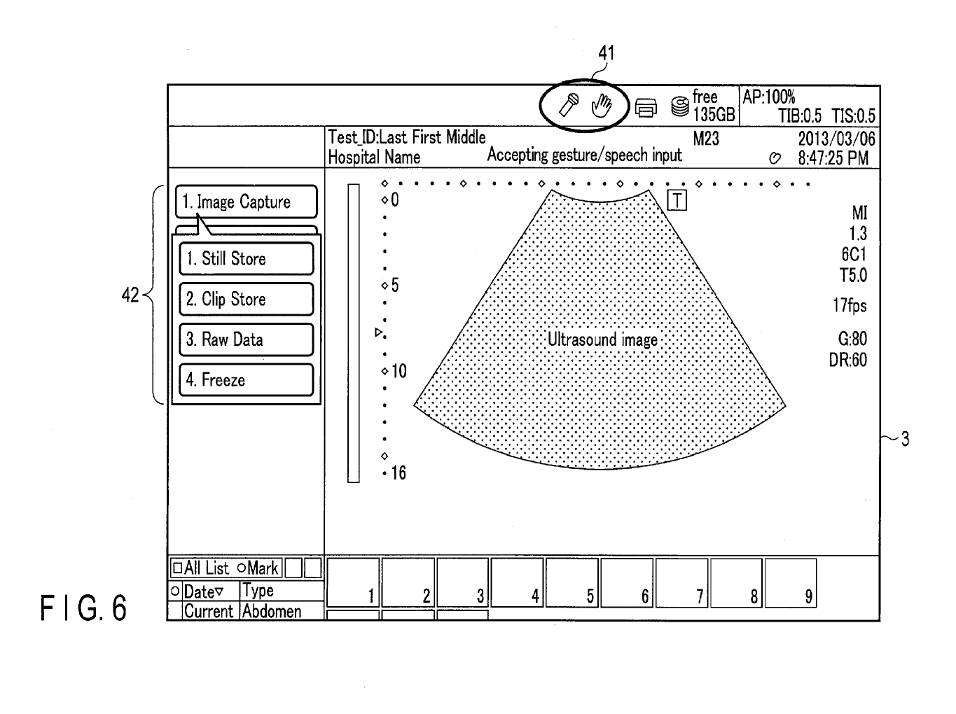

First of all, in step S17, the icon 41 indicating that gesture/speech is being accepted is displayed on the display screen of the monitor 3 after the gesture/speech input acceptance mode is set. In addition, in step S18, target items 42 which can be operated by a gesture/speech input are displayed on the display screen of the monitor 3. FIG. 5 or 6 shows a display example. FIG. 5 shows a case in which category item options are operation targets for a gesture/speech input. FIG. 6 shows a case in which detailed item options in a selected category are operation targets for a gesture/speech input.

Assume that in this state, the operator 7 has raised the number of fingers corresponding to the number of an operation target item by gesture as shown in, for example, FIG. 3. In this case, in steps S19 and S20, the input acceptance processing program 305 extracts an image of the fingers from the image data of the operator imaged by the camera 61, and collates the extracted finger image with a basic image pattern set when a number is expressed by the fingers, which is stored in advance. If the two images match with a degree of similarity equal to or more than a threshold, the number expressed by the finger image is accepted, and a category or detailed item corresponding to the number is selected in step S21.

Assume that the operator has uttered speech representing the number of an operation target item. In this case, the input acceptance processing program 305 performs the processing of detecting the direction of the sound source and speech recognition processing for the speech collected by the microphone 62 in the following manner. That is, beam forming is performed by using the microphone 62 formed from a microphone array. Beam forming is a technique of selectively collecting speech from a specific direction, thereby specifying the direction of the sound source, that is, the direction of the operator. In addition, the input acceptance processing program 305 recognizes a word from the collected speech by using a known speech recognition technique. The input acceptance processing program 305 then determines whether any operation target item corresponding to the word recognized by the above speech recognition technique exists. If such an item exists, the input acceptance processing program 305 accepts the number represented by the word, and selects a category or detailed item corresponding to the number in step S21.

While the above gesture/speech input acceptance mode is set, the input acceptance condition determination program 304 monitors the cancellation of the input acceptance mode in step S22. As long as the state of the operator satisfies the above input acceptance conditions, the gesture/speech input acceptance mode is maintained. In contrast to this, when the operator 7 finishes operating the ultrasound probe 4 or approaches the apparatus and can manually perform an input operation, since the input acceptance conditions are not satisfied, the gesture/speech input acceptance mode is canceled, and the icon 41 is erased.

### Effects of First Embodiment

As described in detail above, in the first embodiment, the use state of the ultrasound probe 4 is determined, and the distance L between the operator and the monitor 3 is calculated after the operator is recognized based on the image data obtained by imaging an examination space using the camera 61. If the ultrasound probe 4 is in use and the distance L is equal to or more than a preset distance, it is determined that the gesture/speech input acceptance conditions are satisfied, and the gesture/speech input acceptance mode is set. A recognition result on a gesture or speech input in this state is accepted as input operation information.

This can limit a gesture/speech input acceptance period to only a period under a situation truly required by the operator. This can prevent the apparatus from recognizing any word or command and performing control corresponding to the word or command regardless of the intention of the operator 7 when the operator 7 unintentionally utters a word or makes a gesture or an operation command is accidentally included in a conversation with an assistant, the object 8, or the like or in an explanation by hand gestures under a situation in which there is no need to perform an input operation by gesture or speech.

In addition, when the gesture/speech input acceptance conditions are satisfied, the icon 41 is displayed on the display screen of the monitor 3, and operation target items are displayed with numbers. This allows the operator 7 to clearly recognize, by seeing the monitor 3, whether the current mode is the mode of enabling a gesture/speech input operation. In addition, the operator can perform a gesture/speech input operation upon checking operation target items.

### Second Embodiment

In the second embodiment, when the operator performs a data operation during a non-examination period to, for example, register, change, or delete patient/examination information, gesture/speech input acceptance conditions are set such that the operator visually recognizes the monitor, and display screen data necessary for the data operation is displayed on the monitor. When the conditions are satisfied, the gesture/speech input acceptance mode is set to execute gesture/speech input acceptance processing.

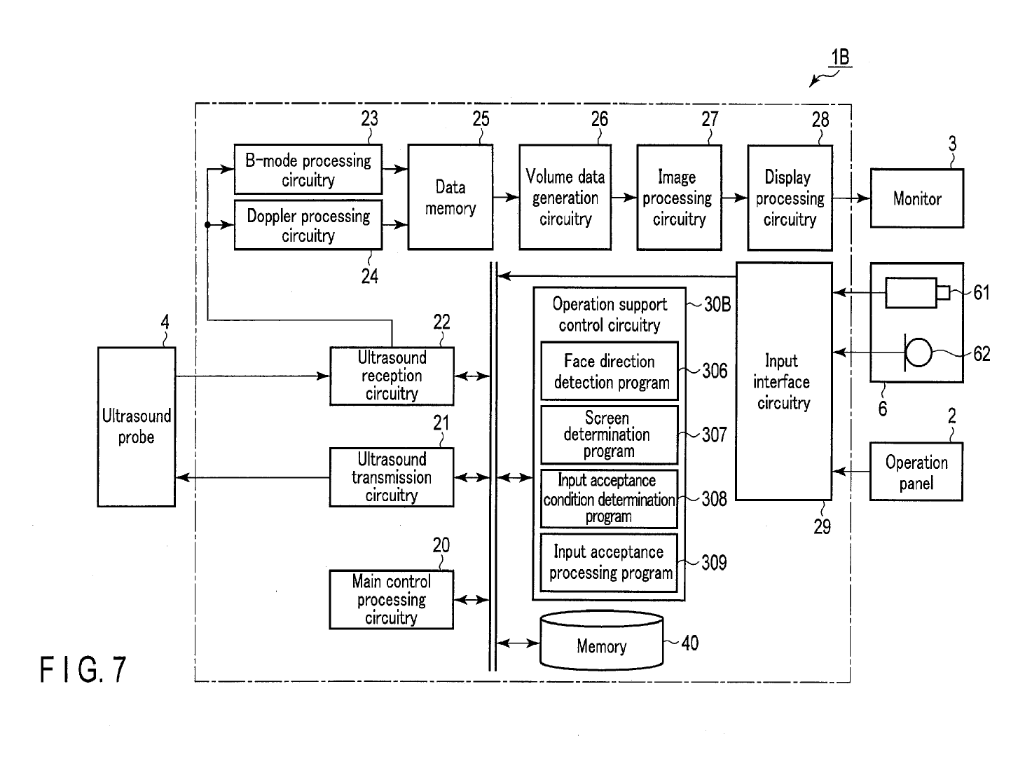

FIG. 7 is a block diagram showing the functional arrangement of an apparatus main body 1B of an ultrasound diagnostic apparatus according to the second embodiment, together with the arrangement of peripheral elements. Note that the same reference numerals as in FIG. 2 denote the same parts in FIG. 7, and a detailed description of them will be omitted.

An operation support control circuitry 30B of the apparatus main body 1B is constituted by, for example, a predetermined processor and a memory. The operation support control circuitry 30B includes a face direction detection program 306, a screen determination program 307, an input acceptance condition determination program 308, and an input acceptance processing program 309 as control functions necessary to execute the second embodiment.

The face direction detection program 306 recognizes a face image of the operator by a pattern recognition technique based on the image data of the operator imaged by a camera 61 of a sensor 6, and determines, based on the recognition result, whether the face of the operator is facing the direction of a monitor 3.

When performing a data operation for control information to, for example, register, change, or delete patient/examination information, the screen determination program 307 determines whether display screen data of a status necessary for the data operation is displayed on the monitor 3.

The input acceptance condition determination program 308 determines, based on the direction of the face of the operator which is detected by the face direction detection program 306 and the status of the display screen data determined by the screen determination program 307, whether the direction of the face of the operator and the status of the display screen data satisfy the gesture/speech operation information input acceptance conditions.

If the input acceptance condition determination program 308 determines that the direction of the face of the operator and the status of the display screen data satisfy the gesture/speech input acceptance conditions, the input acceptance processing program 309 sets the gesture/speech input acceptance mode and displays, on the display screen, an icon indicating that gesture/speech is being accepted. The input acceptance processing program 309 recognizes the gesture and speech of the operator based on the image data of the operator imaged by the camera 61 of the sensor 6 and the speech data of the operator obtained by a microphone 62. The input acceptance processing program 309 then determines the validity of the operation information represented by the recognized gesture and speech. If the operation information is valid, the input acceptance processing program 309 accepts the operation information represented by the gesture and speech.

### (Operation)

The input operation support operation performed by the apparatus having the above arrangement will be described next.

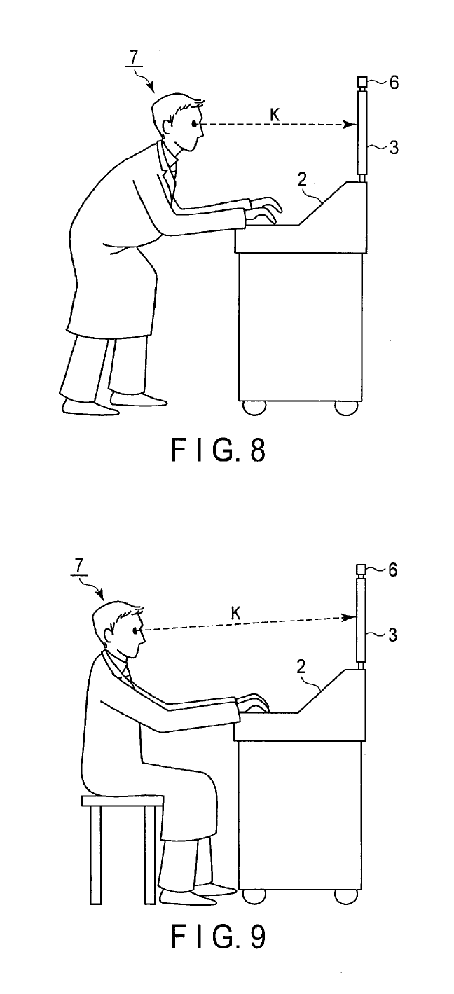

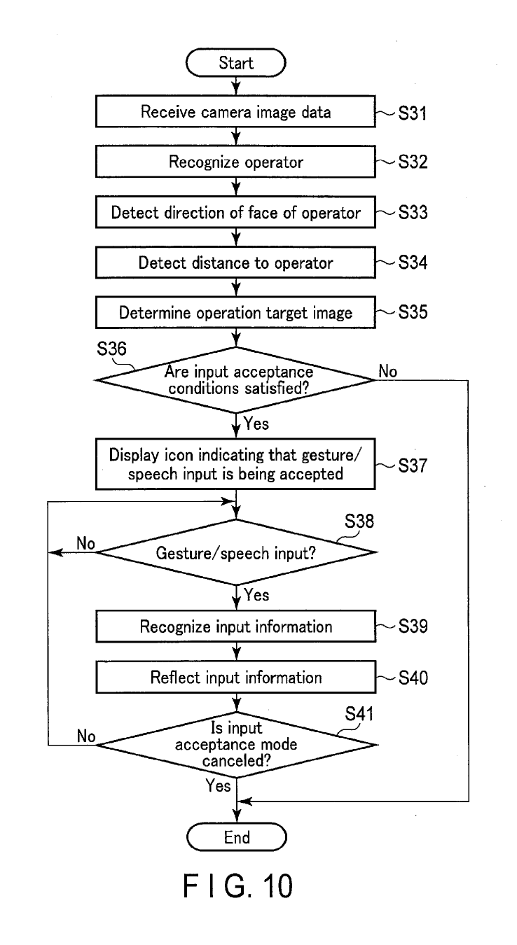

FIGS. 8 and 9 each show an example of the positional relationship between the apparatus and the operator 7. FIG. 8 shows a state in which the operator in a standing position tries to perform an input operation. FIG. 9 shows a state in which the operator in a sitting position tries to perform an input operation. FIG. 10 is a flowchart showing a processing procedure and processing contents of input operation support control executed by the operation support control circuitry 30B.

### (1) Detection of Direction of Face of Operator

The operation support control circuitry 30B executes processing for detecting the direction of the face of the operator under the control of the face direction detection program 306.

That is, first of all, in step S31, the operation support control circuitry 30B receives the image data obtained by imaging an operator 7 from the camera 61 of the sensor 6, and temporarily saves the data in a buffer area in a memory 40. In step S32, the operation support control circuitry 30B recognizes the face image of the operator 7 from the saved image data. This face image is recognized by using a known pattern recognition technique of collating the above obtained image data with the face image pattern of the operator, which is stored in advance. In step S33, an image representing the eyes is extracted from the recognized face image of the operator, and a visual line K of the operator is detected from the extracted eye image.

Subsequently, in step S34, the distance between the monitor 3 of the apparatus and the operator 7 is detected. For example, this distance is detected in the following manner. That is, the sensor 6 irradiates the operator with infrared light, and the light-receiving element of the camera 61 receives the reflected wave generated when the irradiation wave is reflected by the face of the operator 7. The distance is then calculated based on the phase difference between the irradiation wave and the reflected wave or the time from the irradiation to the reception.

### (2) Determination of Operation Target Screen

In step S35, the operation support control circuitry 30B determines, under the control of the screen determination program 307, whether the status of the display screen data displayed on the monitor 3, i.e., the type of display screen and its state, correspond to a case requiring a data operation, based on determination conditions stored in advance. The determination conditions include, for example, the following three states:

(1) a state in which an inquiry is made to a hospital server via a patient information registration screen, examination reservation data has not been registered, and a registering operation is required;
(2) a state in which an editing screen for patient information or examination information is displayed, and a focus is set on a text box of any one of the items in the display screen; and (3) a state in which an examination list is displayed on the display screen, and a focus is set on a keyword input box in the screen.

### (3) Determination on Whether Input Acceptance Conditions are Satisfied

The operation support control circuitry 30B determines in step S36, under the control of the input acceptance condition determination program 308, whether the gesture/speech operation information input acceptance conditions are satisfied, based on the detection result on the direction of the face of the operator (more accurately, a direction K of a visual line) which is obtained by the face direction detection program 306 and the determination result on the status of the display screen data which is obtained by the screen determination program 307.

For example, if the face (visual line) of the operator is facing the monitor 3 and the screen displayed on the monitor 3 corresponds to one of the above states (1), (2), and (3), the operation support control circuitry 30B determines that the gesture/speech input acceptance conditions are satisfied. If this determination indicates that the input acceptance conditions are not satisfied, the operation support control circuitry 30B terminates the input operation support control without setting the gesture/speech operation information input acceptance mode.

Note that as one input acceptance condition, it is possible to add a case in which the distance between the face of the operator 7 and the monitor 3 is within a preset threshold. This makes it possible to inhibit gesture/speech input acceptance by regarding that the operator 7 is not in a state in which he/she can perform a main input operation using an operation panel 2 if the distance between the operator 7 and the monitor 3 exceeds the threshold, even when the display screen data is in a status requiring a data operation or the face of the operator 7 is facing the monitor 3.

### (5) Gesture/Speech Operation Information Input Acceptance Processing

In contrast to this, assume that it is determined in step S36 that the input acceptance conditions are satisfied. In this case, gesture/speech input acceptance processing is executed in the following manner under the control of the input acceptance processing program 309.

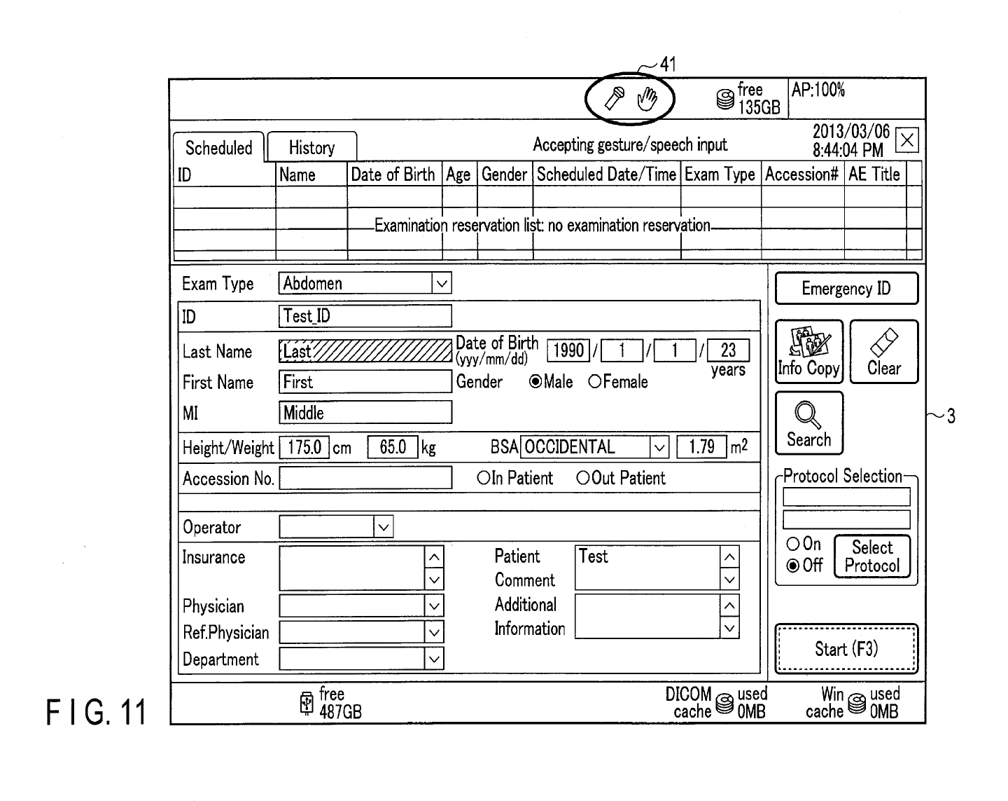

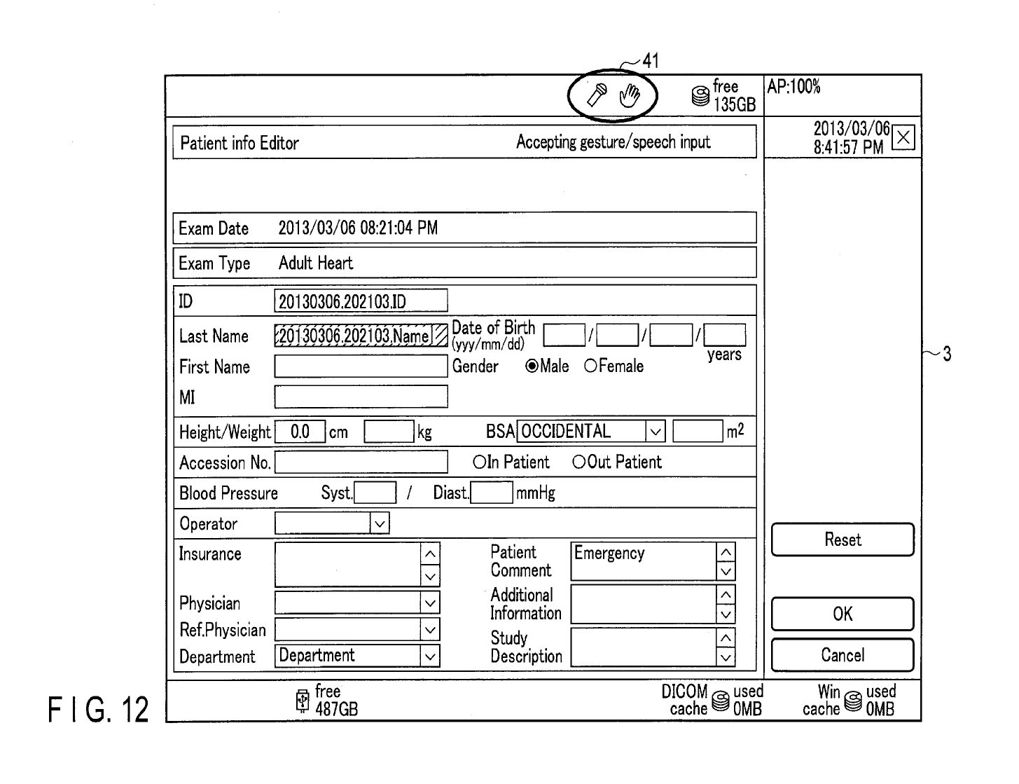

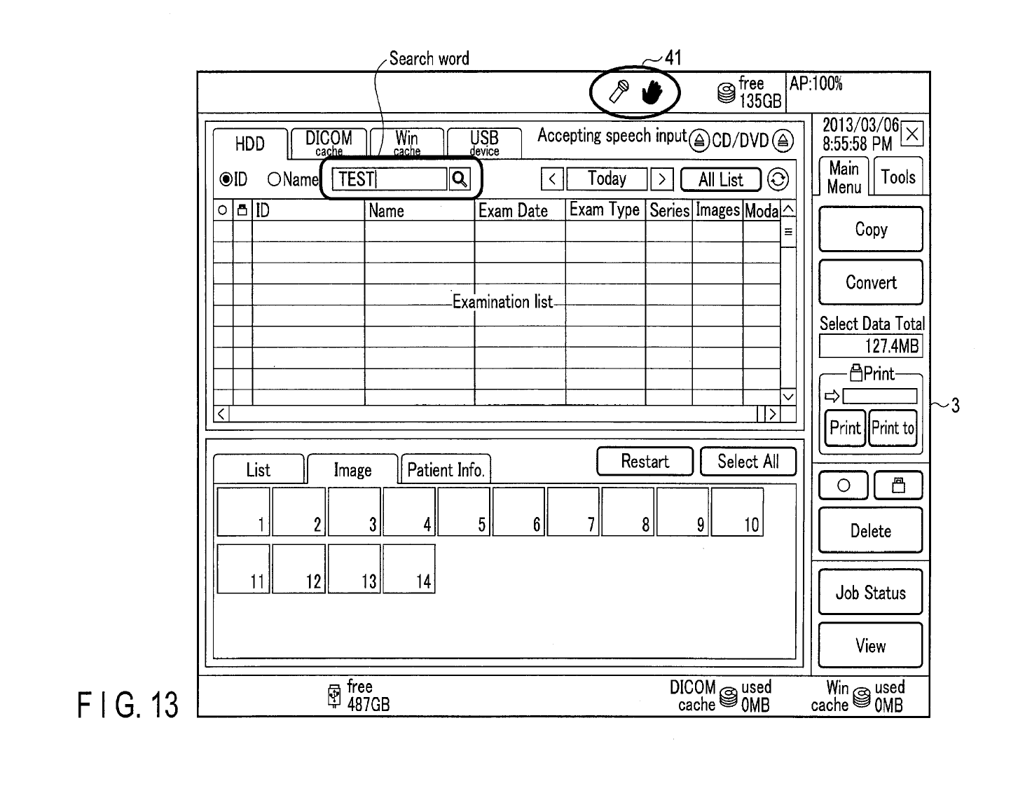

That is, first of all, in step S37, after the gesture/speech input acceptance mode is set, an icon 41 indicating that the mode is currently set is displayed on the display screen of the monitor 3. FIGS. 11, 12, and 13 each show a display example. FIG. 11 shows a case in which the icon 41 is displayed on the patient information registration screen on which no examination reservation information has been registered. FIG. 12 shows a case in which the icon 41 is displayed on the patient/examination information editing screen. FIG. 13 shows a case in which the icon 41 is displayed on the search list display screen.

When the operator 7 inputs operation information by gesture/speech while the gesture/speech input acceptance mode is set, the input acceptance processing program 309 performs operation information input acceptance processing in the following manner.

Assume that the operator 7 has moved his/her finger directed to the display screen of the monitor 3 while the patient information registration screen is displayed as shown in FIG. 11, with no reservation examination information being registered on the reservation examination list. In this case, first of all, in step S39, the input acceptance processing program 309 extracts an image of the fingertip of the operator 7 from the image data obtained by imaging the operator 7 using the camera 61, and detects the moving direction and moving amount of the extracted fingertip image. The input acceptance processing program 309 then moves the position of the focus with respect to the text box in step S40 in accordance with the detection results. When, for example, the operator 7 moves his/her finger downward by gesture by a predetermined amount while the focus is set on the text box “Exam Type”, the gesture is recognized, and the text box on which the focus is set moves to the text box “ID”.

Subsequently, when the operator 7 inputs speech, the microphone 62 detects this input speech. In addition, in step S39, the word input by the operator 7 is recognized from the speech data by known speech recognition processing. In step S40, the word is input to the text box “ID” on which the focus is set.

Assume that as shown in FIG. 12, the operator 7 has moved his/her finger downward by gesture while the patient/examination information editing screen is displayed, and the focus is set on the text box “ID” on the screen, as shown in FIG. 12. In this case, the gesture is recognized, and the text box on which the focus is set moves to the text box “Last Name”. When the operator 7 inputs speech, the microphone 62 detects this input speech. In addition, the word input by the operator 7 is recognized from the speech data by known speech recognition processing. The word is input to the text box “Last Name” on which the focus is set.

In the state in which the examination list is displayed as shown in FIG. 13, only a text box for a search word is the only text box as an input target. For this reason, the icon indicating that gesture is being accepted is not displayed, and only the icon 41 indicating that speech is being accepted is displayed. When the operator 7 inputs a keyword by speech in this state, the microphone 62 detects this input speech. In addition, the keyword input by the operator 7 is recognized from the speech data by known speech recognition processing. The word is input to the text box “search keyword” on which the focus is set.

In this manner, the operator 7 performs a selecting operation for a text box by gesture/speech, and performs the operation of inputting information to the selected text box.

While the gesture/speech input acceptance mode is set, the input acceptance condition determination program 308 monitors the cancellation of the input acceptance mode in step S41. As a result, the gesture/speech input acceptance mode is maintained as long as the state of the operator 7 satisfies the above input acceptance conditions. In contrast to this, assume that the operator 7 averts his/her face from the monitor 3 continuously for a predetermined time or the status of the display screen has changed such that the operator 7 need not perform any data operation. In this case, since the input acceptance conditions are not satisfied, the gesture/speech input acceptance mode is canceled at this point of time, and the icon 41 is also erased.

### Effects of Second Embodiment

As described in detail above, in the second embodiment, when the operator 7 performs a data operation for control information to, for example, register, change, or delete patient/examination information during a non-examination period, the gesture/speech input acceptance mode is set to execute gesture/speech input acceptance processing upon satisfaction of the conditions that the face of the operator 7 is facing the monitor 3, and display screen data necessary for the data operation is displayed on the monitor 3.

When, therefore, the operator 7 tries to perform a data operation for control information to, for example, register, change, or delete patient/examination information, he/she can perform the operation of selecting a text box by gesture/speech and the operation of inputting information to a selected text box. This can improve the operability as compared with a case in which the operator performs all operations with the keyboard and the trackball. In general, the keyboard of the ultrasound diagnostic apparatus is small and needs to be pulled out when used. For this reason, using both an input operation based on the above gesture/speech input operation and an input operation using the keyboard makes it possible to expect a large effect in improving the operability.

In addition, since the above gesture/speech input acceptance conditions are set and an input is accepted only when the conditions are satisfied, it is possible to limit a gesture/speech input acceptance period to only a period under a situation truly required by the operator. This can prevent the apparatus from recognizing any word or command and performing control corresponding to the word or command regardless of the intention of the operator 7 when he/she unintentionally utters a word or makes a gesture or an operation command is accidentally included in a conversation with an assistant, the object 8, or the like or in an explanation by hand gestures under a situation in which there is no need to perform an input operation by gesture or speech.

Furthermore, when gesture/speech input acceptance conditions are satisfied, the icon 41 is displayed on the display screen of the monitor 3, and operation target items are displayed with numbers. This allows the operator 7 to clearly recognize, by seeing the monitor 3, whether the current mode is the mode of enabling a gesture/speech input operation.

### Third Embodiment

The third embodiment is configured to set the gesture input acceptance mode upon determining that gesture/speech input acceptance conditions are satisfied when the face of an operator is facing the direction of the monitor without touching the trackball, with the position of a hand of the operator being higher than that of the panel, while a screen other than that displayed during an examination is displayed and the cursor needs to be moved by an operation on the trackball, recognize the gesture made by the operator in this state, and control the movement of the cursor.

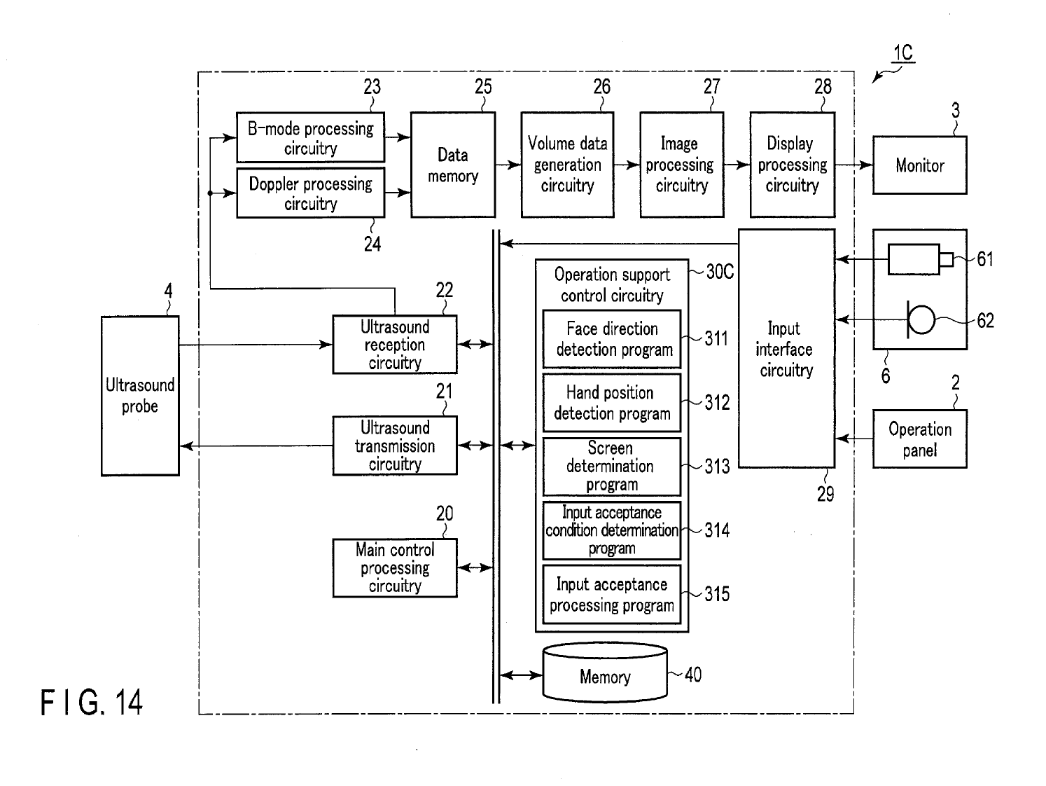

FIG. 14 is a block diagram showing the functional arrangement of an apparatus main body 1C of an ultrasound diagnostic apparatus according to the third embodiment, together with the arrangement of peripheral elements. Note that the same reference numerals as in FIG. 2 denote the same parts in FIG. 14, and a detailed description of them will be omitted.

An operation support control circuitry 30C of the apparatus main body 1C is constituted by, for example, a predetermined processor and a memory. The operation support control circuitry 30C includes a face direction detection program 311, a hand position detection program 312, a screen determination program 313, an input acceptance condition determination program 314, and an input acceptance processing program 315 as control functions necessary to execute the third embodiment.

The face direction detection program 311 recognizes a face image of the operator by a pattern recognition technique based on the image data of the operator imaged by a camera 61 of a sensor 6, and determines, based on the recognition result, whether the face of the operator is facing the direction of a monitor 3.

The hand position detection program 312 recognizes a hand image of the operator by the pattern recognition technique based on the image data of the operator imaged by the camera 61, and determines, based on the recognition result, whether the position of the hand of the operator is higher than that of the operation panel.

The screen determination program 313 determines whether a screen of a type requiring cursor movement like that displayed when patient/examination information is to be browsed is displayed.

The input acceptance condition determination program 314 determines whether the direction of the face of the operator, the height position of the hand of the operator, and the type of display screen satisfy the gesture input acceptance conditions, based on the direction of the face of the operator which is detected by the face direction detection program 311, the position of the hand of the operator which is detected by the hand position detection program 312, and the type of display screen which is determined by the screen determination program 313.

If the input acceptance condition determination program 314 determines that the gesture input acceptance conditions are satisfied, the input acceptance processing program 315 sets the gesture input acceptance mode, and displays, on the display screen, an icon indicating that gesture is being accepted. The gesture of the operator is recognized based on the image data of the operator imaged by the camera 61 of the sensor 6. The validity of the operation information represented by the recognized gesture is determined. If the operation information is valid, the operation information represented by the gesture is accepted, and cursor movement control is performed.

### (Operation)

The input operation support operation of the apparatus having the above arrangement will be described next.

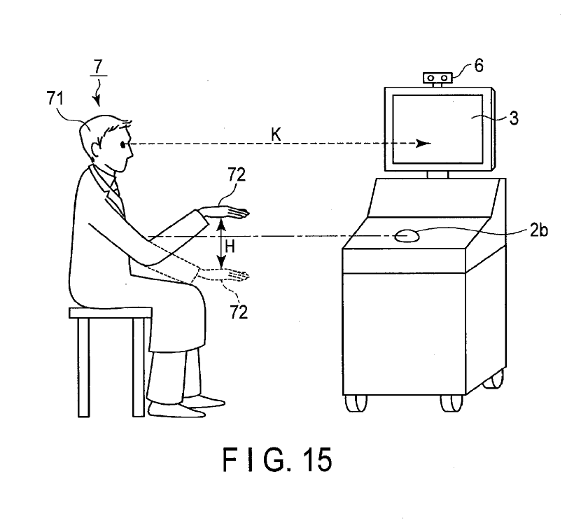

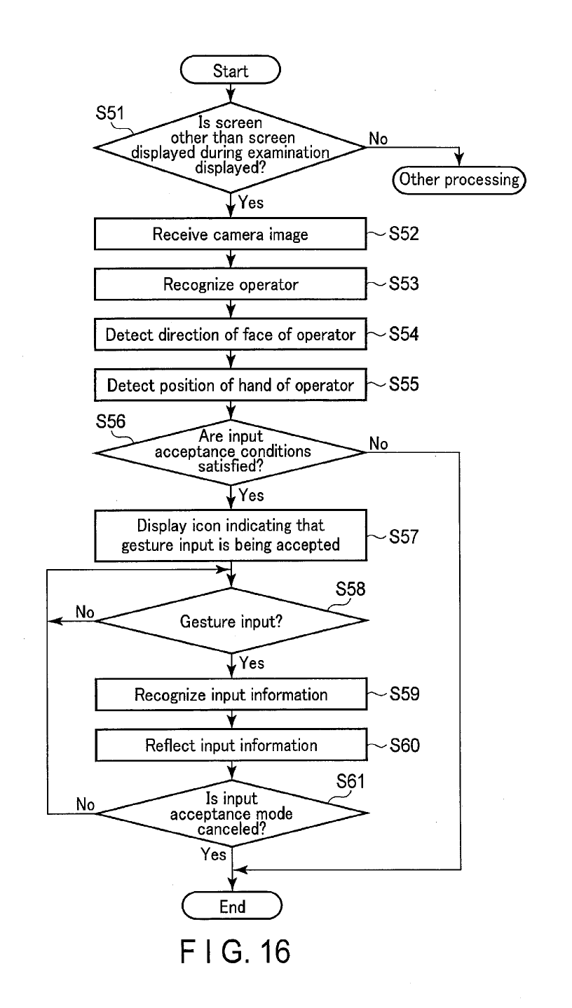

FIG. 15 shows an example of the positional relationship between the apparatus and the operator 7. FIG. 16 is a flowchart showing a processing procedure and processing contents of input operation support control executed by the operation support control circuitry 30C.

### (1) Determination of Display Screen

First of all, in step S51, the operation support control circuitry 30C determines the type of screen displayed on the monitor 3 under the control of the screen determination program 313. In this case, the operation support control circuitry 30C determines whether a screen other than that displayed during an examination is displayed, and a screen requiring a cursor operation like an examination list display screen is currently displayed.

### (2) Detection of Direction of Face of Operator and Position of Hand

The operation support control circuitry 30C executes processing for detecting the direction of the face of an operator in the following manner under the control of the face direction detection program 311.

That is, first all, in step S52, the operation support control circuitry 30C receives the image data obtained by imaging the operator 7 from the camera 61 of the sensor 6, and temporarily saves the data in a buffer area in a memory 40. In step S53, the operation support control circuitry 30C recognizes the face image of the operator 7 from the saved image data. This face image is recognized by using a pattern recognition technique of collating the above obtained image data with the face image pattern of the operator, which is stored in advance. In step S54, an image representing the eyes is extracted from the recognized face image of the operator, and a visual line K of the operator is detected from the extracted eye image.

In step S55, the operation support control circuitry 30C recognizes an image of the hand of the operator 7 from the image data obtained by imaging the operator 7, and determines whether a position H of the recognized hand is higher or lower than a trackball 2b.

### (3) Determination on Whether Input Acceptance Conditions are Satisfied

The operation support control circuitry 30C then determines in step S56, under the control of the input acceptance condition determination program 314, whether the gesture input acceptance conditions are satisfied, based on the detection result on the direction of the face of the operator (more accurately, a direction K of a visual line) which is obtained by the face direction detection program 311, the determination result on the position of the hand of the operator 7 which is obtained by the hand position detection program 312, and the type of currently displayed screen which is determined by the screen determination program 313.

Assume that as shown in FIG. 15, the face of the operator 7 (more accurately, the visual line K) is facing the direction of a monitor 3 and the operator 7 is not touching the trackball 2b, with the position H of the hand of the operator 7 being higher than the position of the operation panel 2, while a screen other than that displayed during an examination is displayed on the monitor 3 and a screen requiring cursor movement by the operation of the trackball 2b is displayed. In this case, it is determined that the gesture input acceptance conditions are satisfied. Note that if it is determined that the gesture input acceptance conditions are not satisfied, the gesture operation information input mode is not set, and the input operation support operation is terminated.

### (4) Gesture Operation Information Input Acceptance Processing

In contrast to this, assume that it is determined in step S56 that the input acceptance conditions are satisfied. In this case, gesture input acceptance processing is executed in the following manner under the control of the input acceptance processing program 315.

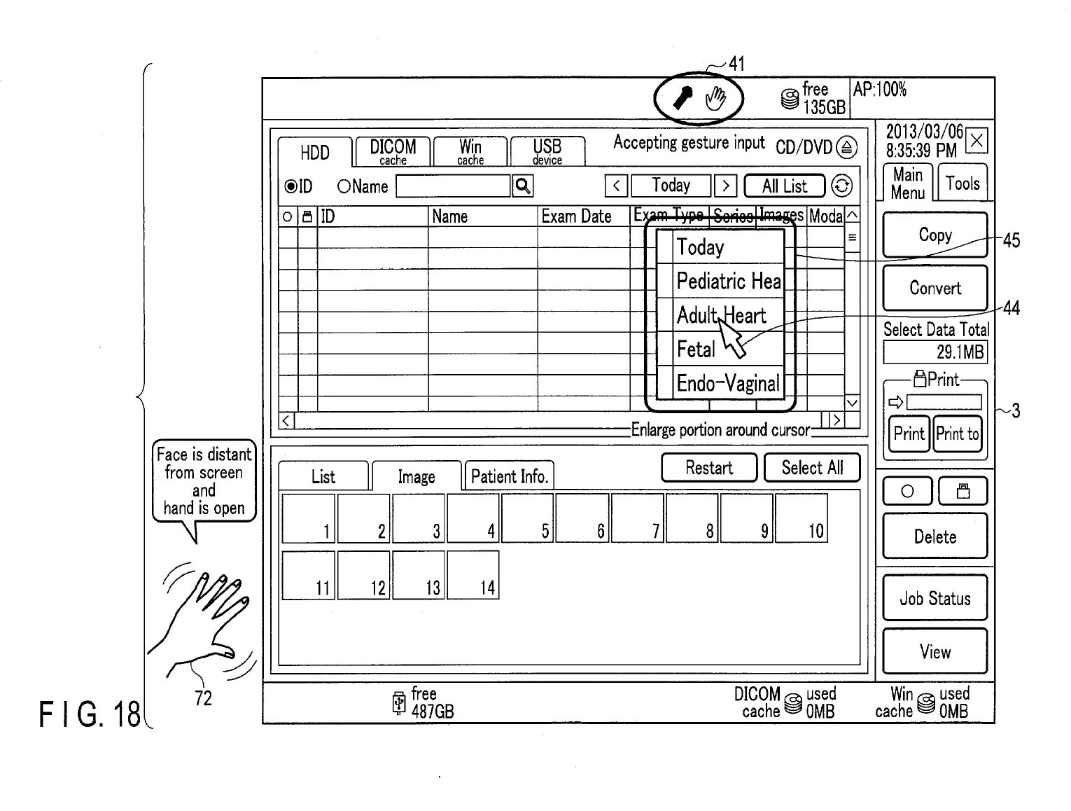

That is, first of all, in step S57, after the gesture input acceptance mode is set, an icon 41 indicating that gesture is being accepted is displayed on the display screen of the monitor 3. FIG. 18 shows an example of the icon and a case in which the icon 41 is displayed on the patient list display screen.

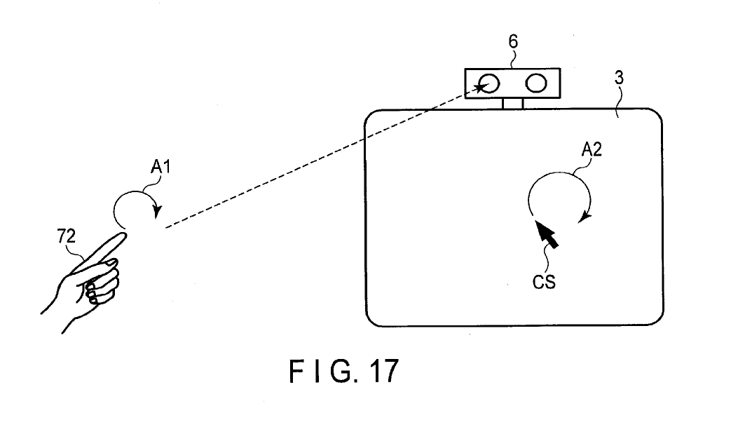

When the operator 7 makes a gesture with his/her finger while the above gesture input acceptance mode is set, the input acceptance processing program 315 performs operation information input acceptance processing in the following manner. That is, assume that the operator 7 has drawn a circle A1 clockwise with his/her finger as shown in FIG. 17. In this case, the input acceptance processing program 315 extracts an image of the finger of the operator 7 from the image data obtained by imaging the operator 7 using the camera 61, and detects the movement of the extracted finger image. Upon detecting that the finger has moved by a predetermined amount or more, the input acceptance processing program 315 determines in step S58 that a gesture has been made, and then recognizes the moving direction and movement amount of the gesture, i.e., the movement locus in step S59. In step S60, the position of a cursor CS currently displayed on the patient list display screen of the monitor 3 is moved as indicated by A2 in FIG. 17 in accordance with this recognized movement locus. In this manner, the cursor CS is moved by the gesture of the operator 7.

While the above gesture input acceptance mode is set, the input acceptance condition determination program 314 monitors the cancellation of the input acceptance mode in step S61. As a result, the above gesture input acceptance mode is maintained as long as the state of the operator 7 satisfies the above input acceptance conditions. In contrast to this, assume that the operator 7 averts his/her face from the monitor 3 continuously for a predetermined time or the type of display screen has changed to a screen requiring no cursor operation by the operator 7 or the operator 7 lowers his/her hand below the operation panel 2. In this case, since the input acceptance conditions are not satisfied, the gesture input acceptance mode is canceled at this point of time, and the icon 41 is also erased.

### Effects of Third Embodiment

As described in detail above, in the third embodiment, when the face of the operator 7 (more accurately, the visual line K) is facing the direction of the monitor 3 and the position H of the hand of the operator 7 is higher than the position of the operation panel 2 without any touch on the trackball 2b while a screen other than that displayed during an examination requiring cursor movement is displayed, since it is estimated that the operator wishes to perform gesture input, it is determined that the gesture input acceptance conditions are satisfied. Subsequently, the gesture input acceptance mode is set, and the icon 41 indicating that gesture is being accepted is displayed on the display screen. The locus of the gesture made by the operator 7 in this state is recognized from image data, thus executing cursor movement processing.

It is therefore possible to move the cursor CS without operating the trackball 2b. In general, the trackball is unsuitable to move the cursor diagonally on a screen. However, since the cursor can be moved by a gesture, the operability can be improved.

In addition, only when the gesture input acceptance conditions are satisfied, i.e., the operator 7 clearly shows his/her intention to perform a cursor operation by gesture input, the gesture input acceptance mode is set. For this reason, when the operator 7 moves his/her finger toward the screen unintentionally or for a different purpose, it is possible to prevent this movement of the finger from being erroneously recognized as a cursor operation.

In addition, when the gesture input acceptance conditions are satisfied, the icon 41 is displayed on the display screen of the monitor 3. This allows the operator 7 to clearly recognize, by seeing the monitor 3, whether the current mode is a mode enabling gesture input.

Note that the third embodiment has exemplified the case in which a cursor operation is performed by gesture. However, this is not exhaustive, and a display screen reducing or enlarging operation may be performed by gesture. FIG. 18 is a view for explaining an example of this operation. When the operator makes a gesture to open his/her hand 72, a predetermined range that includes the information indicated by the cursor is enlarged/displayed.

Gesture input acceptance conditions to be set when executing this example may include, for example, a condition that the distance between the hand 72 of the operator and the monitor 3 is larger than a preset distance and a condition that the character size on the display screen is smaller than a predetermined size. Under such conditions, when the operator experiences difficulty in reading the characters displayed on the screen because he/she is located far from the monitor 3 or because the display character size is small even if he/she is located close to the monitor 3, since a predetermined range that includes the information designated by the cursor is enlarged/displayed, it is possible to make characters included in the range more readable.

### Fourth Embodiment

The fourth embodiment is configured to determine that an operator is seeing the monitor when the operator is operating an ultrasound probe during an examination or is facing the monitor continuously for a predetermined time within a predetermined distance from the monitor even during a non-examination period, activate a display direction tracking control function, and perform control to make the display direction of the monitor track the direction of the face of the operator.

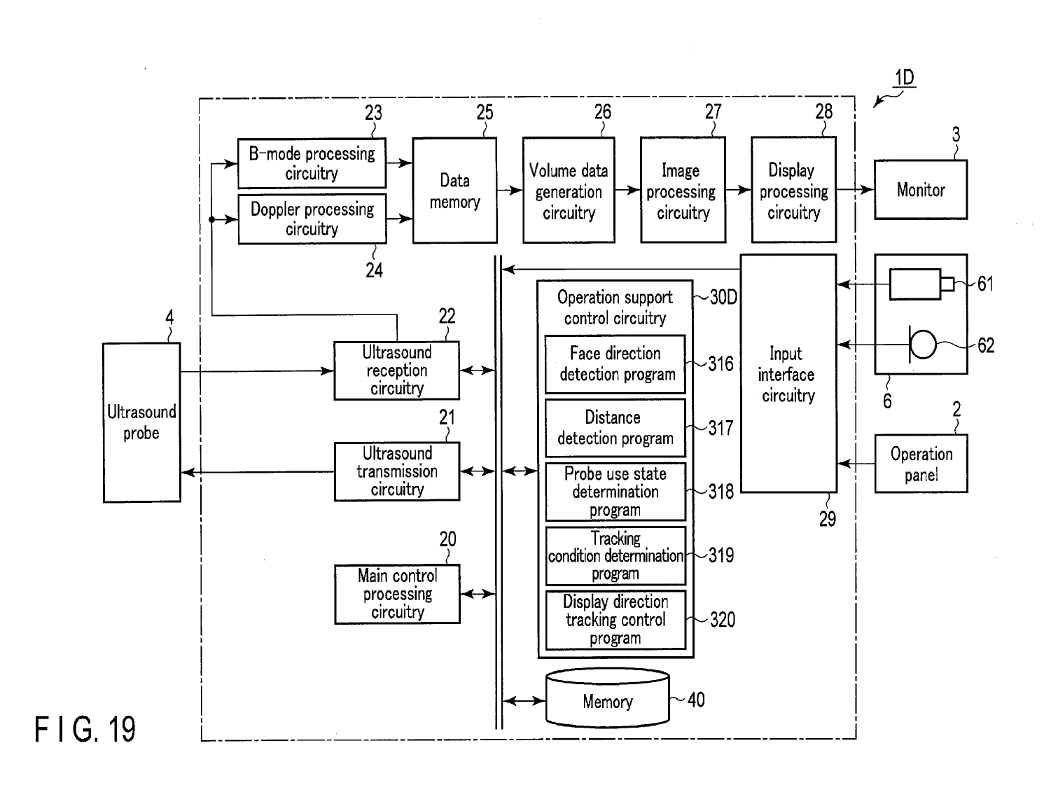

FIG. 19 is a block diagram showing the functional arrangement of an apparatus main body 1D of an ultrasound diagnostic apparatus according to the fourth embodiment, together with the arrangement of peripheral elements. Note that the same reference numerals as in FIG. 2 denote the same parts in FIG. 19, and a detailed description of them will be omitted.

An operation support control circuitry 30D of the apparatus main body 1D is constituted by, for example, a predetermined processor and a memory. The operation support control circuitry 30D includes a face direction detection program 316, a distance detection program 317, a probe use state determination program 318, a tracking condition determination program 319, and a display direction tracking control program 320 as control functions necessary to execute the fourth embodiment.

The face direction detection program 316 recognizes a face image of the operator by a pattern recognition technique based on the image data of the operator imaged by a camera 61 of a sensor 6, and determines, based on the recognition result, whether the face of the operator is facing the direction of a monitor 3.

The distance detection program 317 uses, for example, the distance measurement light source and its light-receiving element of the camera 61 of the sensor 6 to irradiate the operator with infrared light from the light source and receive reflected light by the light-receiving element, and calculates the distance between an operator 7 and the monitor 3 based on the phase difference between the received reflected light and the irradiated light or the time from the irradiation to the reception.

The probe use state determination program 318 determines whether an ultrasound probe 4 is in use, depending on whether a main control processing circuitry 20 is under the examination mode or a live ultrasound image is displayed on the monitor 3.

Based on the detection result on the direction of the face of the operator 7 which is obtained by the face direction detection program 316, the detection result on the distance between the operator 7 and the monitor 3 which is obtained by the distance detection program 317, and the determination result on the use state of the ultrasound probe 4 which is obtained by the probe use state determination program 318, the tracking condition determination program 319 determines whether the detection results or the determination result satisfies preset display direction tracking conditions for the monitor 3.

If the tracking condition determination program 319 determines that the respective detection results and the determination result satisfy the display direction tracking conditions, the display direction tracking control program 320 performs control to make the display direction of the monitor 3 always follow the direction of the face of the operator 7 based on the detection result on the face direction of the operator which is obtained by the face direction detection program 316.

### (Operation)

The input operation support operation of the apparatus having the above arrangement will be described next.

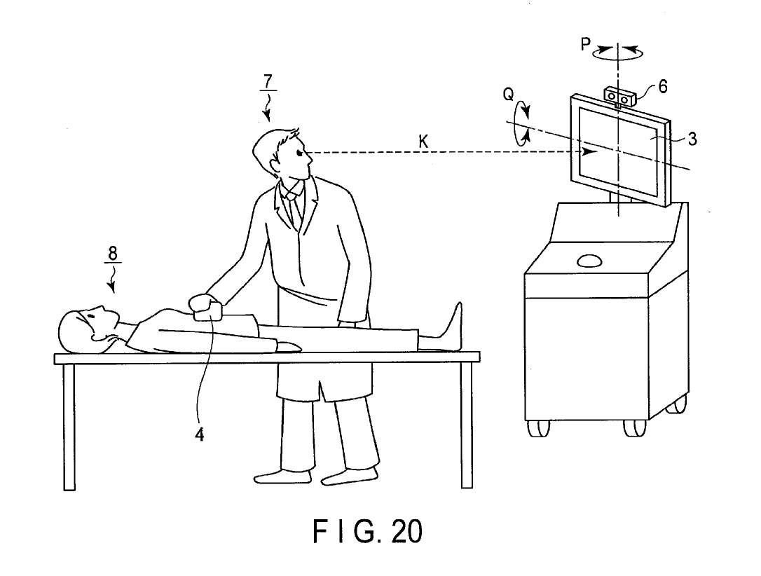

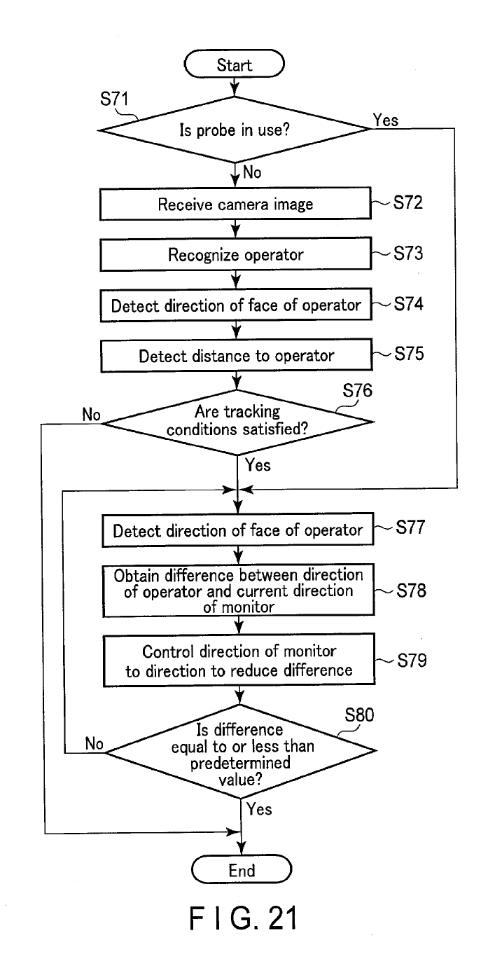

FIG. 20 shows an example of the positional relationship between the apparatus main body 1D and the operator 7 FIG. 21 is a flowchart showing a processing procedure and processing contents of input operation support control executed by the operation support control circuitry 30D.

### (1) Determination of Use State of Ultrasound Probe 4

First of all, in step S71, the operation support control circuitry 30D determines, under the control of the probe use state determination program 318, whether the ultrasound probe 4 is in use. This determination can be made depending on whether the main control processing circuitry 20 is under the examination mode or a live ultrasound image is displayed on the monitor 3.

### (2) Detection of Direction of Face of Operator and Distance

The operation support control circuitry 30D executes processing for detecting the direction of the face of an operator in the following manner under the control of the face direction detection program 316.

That is, first of all, in step S72, the operation support control circuitry 30D receives the image data obtained by imaging the operator 7 from the camera 61 of the sensor 6, and temporarily saves the data in a buffer area in a memory 40. In step S73, the operation support control circuitry 30D recognizes the face image of the operator 7 from the saved image data. This face image is recognized by using a known pattern recognition technique of collating the above obtained image data with the face image pattern of the operator, which is stored in advance. In step S74, an image representing the eyes is extracted from the recognized face image of the operator, and a visual line K of the operator is detected from the extracted eye image.

### (3) Detection of Distance Between Monitor and Operator

Subsequently, in step S75, the operation support control circuitry 30D detects the distance between the monitor 3 and the operator 7 under the control of the distance detection program 317. For example, this distance is detected in the following manner. That is, as described above, the distance measurement light source and its light-receiving element of the camera 61 are used to irradiate the operator with infrared light from the light source and receive reflected light by the light-receiving element. The distance between the operator 7 and the monitor 3 is then calculated based on the phase difference between the received reflected light and the irradiated light or the time from the irradiation to the reception.

### (4) Determination of Tracking Conditions

The operation support control circuitry 30D determines in step S76, under the control of the tracking condition determination program 319, whether the detection result on the direction of the face of the operator 7 which is obtained by the face direction detection program 316, the detection result on the distance between the operator 7 and the monitor 3 which is obtained by the distance detection program 317, and the determination result on the use state of the ultrasound probe 4 which is obtained by the probe use state determination program 318 satisfy preset tracking conditions.

Display direction tracking conditions are set, for example, as follows:

(1) the ultrasound probe 4 is used during an examination; and
(2) the operator 7 exists within a preset distance (e.g., 2 m) from the monitor 3, and the face of the operator 7 is facing the direction of the monitor 3 continuously for a predetermined time (e.g., 2 sec) during a non-examination period.

### (5) Display Direction Tracking Control

Upon determining in step S76 that the detection result on the direction of the face of the operator 7 which is obtained by the face direction detection program 316, the detection result on the distance between the operator 7 and the monitor 3 which is obtained by the distance detection program 317, and the determination result on the use state of the ultrasound probe 4 which is obtained by the probe use state determination program 318 satisfy the above display direction tracking conditions, the operation support control circuitry 30D controls the display direction of the monitor 3 in the following manner under the control of the display direction tracking control program 320.

That is, in step S77, the operation support control circuitry 30D causes the face direction detection program 316 to detect the direction of the face of the operator 7 when viewed from the monitor 3 (in practice, the sensor 6) as a coordinate position on the two-dimensional coordinate system defined in an examination space. In step S78, the operation support control circuitry 30D then calculates the differences between coordinate values representing the detected direction of the face of the operator 7 and coordinate values representing the current display direction of the monitor 3 along the X- and Y-axes, respectively. In step S79, the operation support control circuitry 30D calculates variable angles in a pan direction P and a tilt direction Q of the monitor 3 in accordance with the calculated differences along the X- and Y-axes, and controls the direction of the screen of the monitor 3 by driving a support mechanism for the monitor 3 in accordance with the calculated variable angels. After this direction control, the operation support control circuitry 30D calculates the differences again, and determines in step S80 whether the differences become equal to or less than predetermined values. If this determination result indicates that the differences become equal to or less than the predetermined values, the tracking control is terminated. If the determination result indicates that the differences do not become equal or less than the predetermined values, the process returns to step S77 to repeat tracking control in steps S77 to S80 described above.

### Effects of Fourth Embodiment

As described in detail above, according to the fourth embodiment, when the operator 7 is using the ultrasound probe 4 during an examination or exists within a preset distance (e.g., 2 m) from the monitor 3 during a non-examination period and the face of the operator 7 is facing the direction of the monitor 3 continuously for a predetermined time (e.g., 2 sec), the tracking mode for the direction of the screen of the monitor 3 is set, and tracking control is performed to make the direction of the screen of the monitor 3 always follow the direction of the face of the operator in accordance with the detection result on the position of the face of the operator 7.

Even if, therefore, the position or posture of the operator 7 changes during the operation of the ultrasound probe 4, the operator 7 need not manually correct the direction of the screen of the monitor 3, resulting in an improvement in examination efficiency. This effect is especially effective when the two hands of the operator 7 are occupied during, for example, a surgical operation.

### Fifth Embodiment

When performing a catheter surgery typified by, for example, a cardiovascular surgery, the surgeon sometimes monitors the inside of an object by using an ultrasound diagnostic apparatus. In a cardiovascular surgery, in particular, importance is attached to evaluation based on TEE (transesophageal echocardiography). However, the surgeon performs this surgery under an environment in which various types of apparatuses such as an X-ray diagnostic apparatus, and extracorporeal circulation apparatus are installed in addition to the ultrasound diagnostic apparatus. That is, the ultrasound diagnostic apparatus needs to be operated in a limited space (in general, when obtaining an ultrasound image in a cardiovascular surgery or the like, the technician needs to insert a transesophageal echocardiography probe into the esophagus or stomach of a patient through his/her mouth and obtain an ultrasound image concerning the heart from the inside of the body while standing in a limited place so as not to interfere with the catheter operation of the surgeon and changing his/per posture in the standing position). In such a case, it is expected that the technician may experience difficulty in operating the ultrasound diagnostic apparatus.

The fifth embodiment will therefore exemplify a case in which the technician who assists the surgeon remotely operates the ultrasound diagnostic apparatus. In addition, the surgeon is allowed to perform a gesture/speech input operation, as well as the technician, by becoming an operator by, for example, inputting a predetermined phrase such as “I'm an operator” by speech (in other words, by acquiring the right to operate an ultrasound diagnostic apparatus 1E).

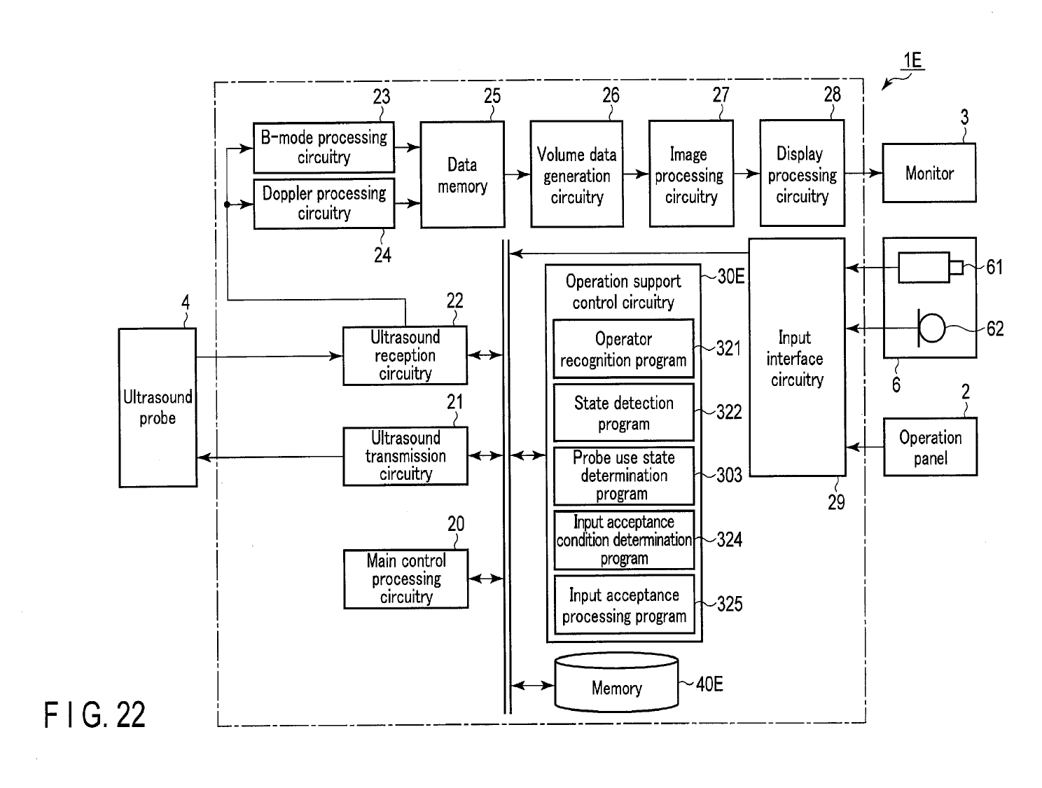

FIG. 22 is a block diagram showing the functional arrangement of the apparatus main body 1E of the ultrasound diagnostic apparatus according to the fifth embodiment, together with the arrangement of peripheral elements. Note that the same reference numerals as in FIG. 2 denote the same parts in FIG. 22, and a detailed description of them will be omitted.

An operation support control circuitry 30E of the apparatus main body 1E is constituted by, for example, a predetermined processor and a memory. The operation support control circuitry 30E includes an operator recognition program 321, a state detection program 322, a probe use state determination program 303, an input acceptance condition determination program 324, and an input acceptance processing program 325 as control functions necessary to execute the fifth embodiment.

The operator recognition program 321 discriminates a surgeon by comparing a person existing in an examination space with the image data of the surgeon registered in advance in a memory 40E based on the image data of the examination space saved in the memory 40E.

Image data for identifying a surgeon who performs a surgical operation is registered in advance in the memory 40E of the apparatus main body 1E in addition to the information saved in the memory 40 in the first embodiment. In addition, in the memory 40E of the apparatus main body 1E, the image pattern of an ultrasound probe 4 stored in advance includes a pattern in which the probe main body portion is partly hidden when the ultrasound probe 4 is inserted into the object 8 through the mouth for a transesophageal echocardiography examination.

The state detection program 322 detects whether the ultrasound probe 4 has been inserted into the object 8 through the mouth, instead of the distance L detected by the distance detection program 302 according to the first embodiment, based on the image data obtained by a camera 61 of a sensor 6. Note that it is possible to detect whether the ultrasound probe 4 has been inserted into the object 8 through the mouth, by using the ultrasound image displayed on the monitor 3.

The input acceptance condition determination program 324 determines whether a probe operating state (imaging state) in which an operator is operating a ultrasound probe satisfies gesture/speech input acceptance conditions, based on the inserted state of the ultrasound probe 4 into the object 8 through the mouth which is detected by the state detection program 322, and the use state of the ultrasound probe 4 determined by the probe use state determination program 303.

The input acceptance processing program 325 sets the gesture input acceptance mode and displays, on the display screen of the monitor 3, an icon indicating that gesture/speech is being accepted, when the input acceptance condition determination program 324 determines that the probe operating state satisfies the gesture/speech operation information input acceptance conditions.

The input acceptance processing program 325 respectively recognizes the gesture and speech of a technician 9 from the image data of the technician 9 obtained by the camera 61 of the sensor 6 and the speech data of the technician 9 obtained by a microphone 62. The input acceptance processing program 325 determines the validity of the operation information represented by the recognized gesture and speech, and accepts the operation information represented by the gesture and speech if the information is valid.

If the input acceptance condition determination program 324 determines that the current probe operating state satisfies the gesture/speech operation information input acceptance conditions, the input acceptance processing program 325 respectively recognizes the gesture and speech of a surgeon 10 from the image data of the surgeon 10 obtained by the camera 61 of the sensor 6 and the speech data of the surgeon 10 obtained by the microphone 62. Upon detecting that a sign from the surgeon 10, which indicates that he/she wishes to become an operator, the input acceptance processing program 325 sets a multiple operator gesture input acceptance mode and displays, on the display screen of the monitor 3, an icon indicating that gesture/speech input acceptance from the technician 9 and the surgeon 10 is ready. The input acceptance processing program 325 accepts the operation information represented by a gesture and speech from both the surgeon 10 and the technician 9 in the same manner.

### (Operation)

The input operation support operation of the apparatus having the above arrangement will be described next.

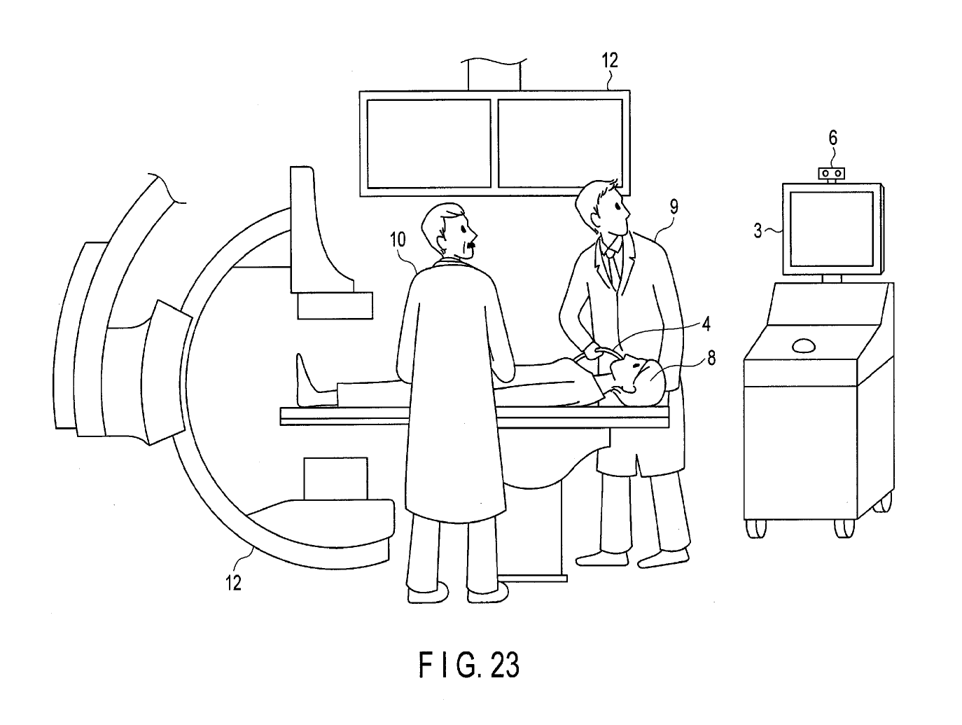

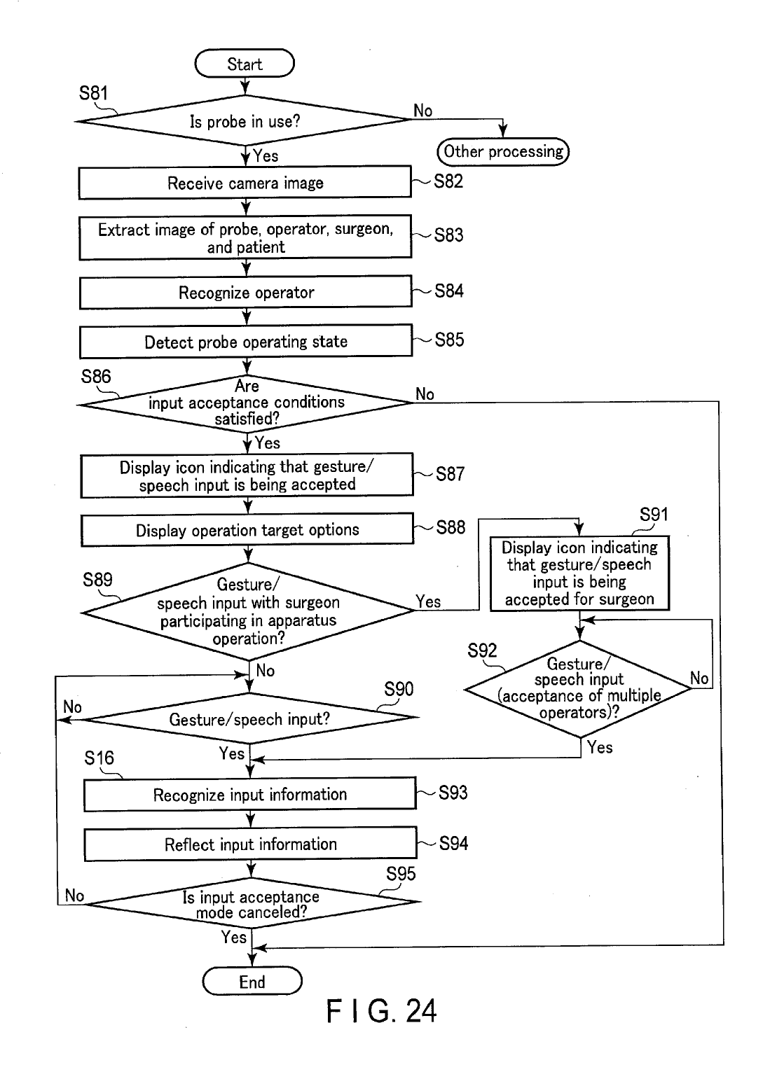

FIG. 23 is a view showing an example of the positional relationship between the apparatus main body 1E and the ultrasound probe 4, the object 8, the technician 9 as an assistant for a surgical operation, the surgeon 10, and an X-ray diagnostic apparatus 12. FIG. 24 is a flowchart showing a processing procedure and processing contents of the input operation support control executed by the operation support control circuitry 30E.

### (1) Determination of Use State of Ultrasound Probe 4

First of all, in step S81, the operation support control circuitry 30E determines, under the control of the probe use state determination program 303, whether the ultrasound probe 4 is in use. This determination can be made depending on whether the main control processing circuitry 20 is under the examination mode or a live ultrasound image is displayed on the monitor 3.

### (2) Recognition of Operator

The operation support control circuitry 30E then executes processing for recognizing the operator in the following manner under the control of the operator recognition program 321.

That is, first of all, in step S82, the operation support control circuitry 30E receives the image data obtained by imaging the examination space from the camera 61 of the sensor 6, and saves the data in a buffer area in the memory 40E. In step S83, the operation support control circuitry 30E then recognizes an image of the ultrasound probe 4 and the person from the saved image data. The recognition of the ultrasound probe 4 is performed by using, for example, pattern recognition. More specifically, with respect to the saved 1-frame image data (in this case, the image data obtained by imaging a state in which a transesophageal echocardiography probe is inserted into the mouth of the patient), a target region of a smaller size is set. Every time this target region is shifted by one pixel, the resultant image is collated with an image pattern stored in advance (for example, the image data obtained in advance by imaging a state in which a transesophageal echocardiography probe is inserted into the mouth of the patient). When the degree of matching becomes equal to or more than a threshold, the collation target image is recognized as an image of the ultrasound probe 4. In step S14, the person holding the ultrasound probe 4 extracted in the above manner is recognized as an operator (in this case, the technician 9).

### (3) Detection of Probe Operating State

In step S85, the state detection program 322 then detects whether the ultrasound probe 4 has been inserted into the object 8 through the mouth. Note that the distance or the like between the technician 9 and the monitor 3 may be detected as needed.

More specifically, the sensor 6 acquires an image of the ultrasound probe 4 and the object 8 obtained by the camera 61. The state detection program 322 then detects, based on the positional relationship between the ultrasound probe 4 and the object 8 depicted on the image, whether the ultrasound probe 4 has been inserted into the object 8 through the mouth.

### (4) Determination on Whether Input Acceptance Conditions are Satisfied

After it is detected whether the ultrasound probe 4 has been inserted into the object 8 through the mouth, it is determined in step S86, under the control of the input acceptance condition determination program 324, whether the current state of the technician 9 satisfies the gesture/speech operation information input acceptance conditions, based on the detection result on whether the ultrasound probe 4 has been inserted into the object 8 through the mouth, which is obtained by the state detection program 322, and the determination result on the use state of the ultrasound probe 4 which is determined by the probe use state determination program 303. Assume that the ultrasound probe 4 is in use in step S81, and the ultrasound probe 4 has been inserted into the object 8 through the mouth. In this case, it is determined that the input acceptance conditions are satisfied. If the determination result indicates that the input acceptance conditions are not satisfied, the input acceptance condition determination program 324 terminates the input operation support control without setting the gesture/speech operation information input acceptance mode.

### (5) Gesture/Speech Operation Information Input Acceptance Processing

In contrast to this, assume that it is determined in step S86 that the input acceptance conditions are satisfied. In this case, gesture/speech input acceptance processing is executed in the following manner under the control of the input acceptance processing program 325.

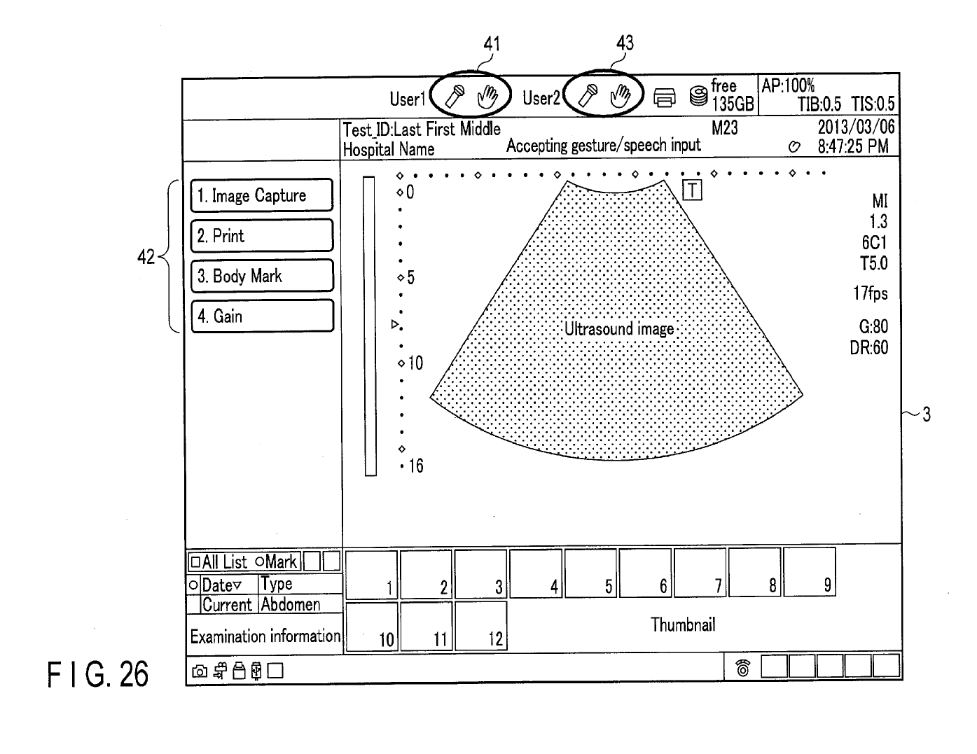

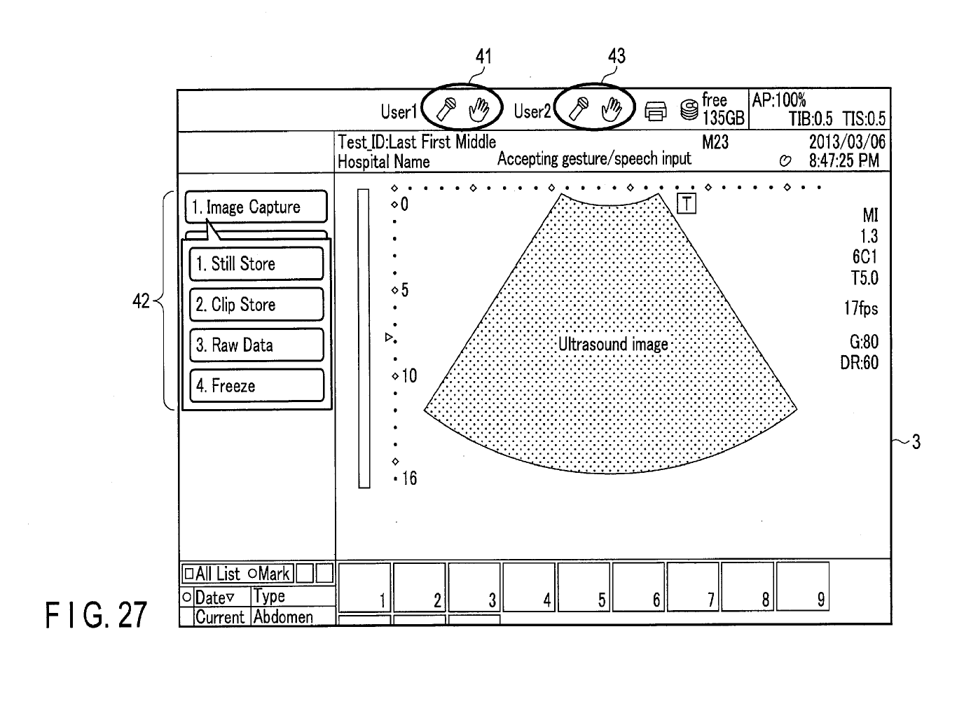

That is, first of all, in step S87, after the gesture input acceptance mode is set, an icon 41 indicating that a gesture/speech input from the technician 9 is being accepted is displayed on the display screen of the monitor 3. In addition, in step S88, target items 42 which can be operated by a gesture/speech input are displayed on the display screen of the monitor 3. FIGS. 26 and 27 each show a display example. FIG. 26 shows a case in which category item options are operation targets for a gesture/speech input. FIG. 27 shows a case in which detailed item options in a selected category are operation targets for a gesture/speech input.

In step S89, the input acceptance processing program 325 then accepts a gesture/speech input from the surgeon 10, which indicates that he/she wants to operate the apparatus. Upon reception of the above gesture/speech input from the surgeon 10, in step S91, the input acceptance processing program 325 displays, on the display screen of the monitor 3, an icon 43 indicating that a gesture/speech input from the surgeon 10 is being accepted. Thereafter, the input acceptance processing program 325 stands by to accept gesture/speech inputs from both the technician 9 and the surgeon 10 in step S92. If there is no gesture/speech input from the surgeon 10, which indicates that he/she wants to operate the apparatus, the input acceptance processing program 325 stands by to accept a gesture/speech input only from the technician 9 in step S90. The following is a case in which the technician 9 performs a gesture/speech input.

Assume that the technician 9 has raised the number of fingers corresponding to the number of an operation target item by gesture as shown in, for example, FIG. 3. In this case, the input acceptance processing program 325 extracts in steps S90 and S93, an image of the fingers from the image data of the operator imaged by the camera 61, and collates the extracted finger image with a basic image pattern set when a number is expressed by the fingers, which is stored in advance. If the two images match with a degree of similarity equal to or more than a threshold, the input acceptance processing program 325 accepts the number expressed by the finger image, and selects a category or detailed item corresponding to the number in step S94.

Assume that the technician 9 has uttered speech representing the number of an operation target item. In this case, the input acceptance processing program 325 performs the processing of detecting the direction of the sound source and speech recognition processing in the following manner with respect to the speech collected by the microphone 62. That is, beam forming is performed by using the microphone 62 formed from a microphone array. Beam forming is a technique of selectively collecting speech from a specific direction, thereby specifying the direction of the sound source, that is, the direction of the technician 9. In addition, the input acceptance processing program 325 recognizes a word from the collected speech by using a known speech recognition technique. The input acceptance processing program 325 then determines whether any operation target item corresponding to the word recognized by the above speech recognition technique exists. If such an item exists, the input acceptance processing program 325 accepts the number represented by the word, and selects a category or detailed item corresponding to the number in step S94.

While the above gesture/speech input acceptance mode is set, the input acceptance condition determination program 324 monitors the cancellation of the input acceptance mode in step S95. As long as the state of the technician satisfies the above input acceptance conditions, the input acceptance condition determination program 324 maintains the gesture/speech input acceptance mode. In contrast to this, when the technician 9 finishes operating the ultrasound probe 4 or approaches the apparatus and can manually perform an input operation, since the input acceptance conditions are not satisfied, the input acceptance condition determination program 324 cancels the gesture/speech input acceptance mode, and erases the icon 41.

### Effects of Fifth Embodiment

As described in detail above, in the fifth embodiment, the use state of the ultrasound probe 4 is determined, and the technician 9 is recognized based on the image data obtained by imaging the examination space using the camera 61. It is then detected whether the ultrasound probe 4 has been inserted into the object 8 through the mouth. If it is determined that the ultrasound probe 4 is in use and it is detected that the ultrasound probe 4 has been inserted into the object 8 through the mouth, the input acceptance condition determination program 324 determines that the gesture/speech input acceptance conditions are satisfied. Upon determining that the gesture/speech input acceptance conditions are satisfied, the input acceptance condition determination program 324 sets the gesture/speech input acceptance mode, and accepts a recognition result on a gesture or speech input in this state as input operation information. In addition, the input acceptance processing program 325 accepts a gesture/speech input from the surgeon 10 indicating a desire to operate the apparatus, and is ready to accept input operation information not only from the technician 9 but also from the surgeon 10.

This can limit a gesture/speech input acceptance period to only a period under a situation truly required by the operator. This can prevent the apparatus from recognizing any word or command and performing control corresponding to the word or command regardless of the intention of the technician 9 when the technician 9 unintentionally utters a word or makes a gesture or an operation command is accidentally included in a conversation with the object 8, the surgeon 10, or the like or in an explanation by hand gestures under a situation in which there is no need to perform an input operation by gesture or speech. In addition to a person who detects and other than the technician 9, for example, the surgeon 10 can participate in an operation. This makes it possible to improve examination or surgery efficiency.

In addition, when the gesture/speech input acceptance conditions are satisfied, the icons 41 and 43 are displayed on the display screen of the monitor 3, and operation target items are displayed with numbers. This allows the technician 9 and the surgeon 10 to clearly recognize, by seeing the monitor 3, whether the current mode is the mode of enabling a gesture/speech input operation. In addition, they can perform an input operation by gesture/speech upon checking operation target items.

### First Modification

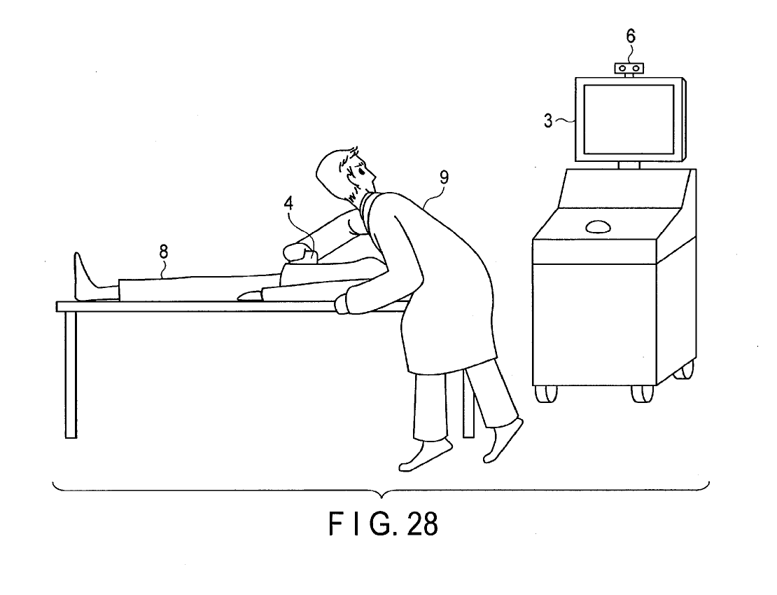

As a case in which a technician needs to operate an ultrasound probe in a limited posture, the first modification will exemplify a case in which the technician performs an examination on the opposite (back) side of a lying patient to the technician. FIG. 28 is a view for explaining the first modification of the fifth embodiment, showing an example of the positional relationship between the apparatus and the operator.

When performing an examination using an ultrasound probe in a limited narrow space such as a patient's room, for example, the technician 9 sometimes stands on the left side of the object 8 and brings the ultrasound probe 4 into contact with the chest portion on the right side, which is the opposite (back) side to the technician 9, across the body of the object 8 while bending his/her body so as to lean over the body of the object 8. In such a case, the apparatus detects the unnatural posture of the technician 9 by detecting a body axis angle θ of the technician 9 who has performed the above probe bringing operation, and performs control as one of the gesture/speech input acceptance conditions.

The following three detection items are used as gesture/speech input acceptance conditions in the first modification: the distance between the technician 9 and the monitor 3; the body axis angle of the technician 9 relative to the vertical direction (the barycentric direction or the direction perpendicular to the floor); and the contact/non-contact of the ultrasound probe 4 operated by the technician 9 with respect to the object 8. The first modification is configured to detect the following three items instead of the items detected by the state detection program 322 in FIG. 22.

### (a) Detection of Distance L Between Technician 9 and Monitor 3

The distance L between the monitor 3 and a specific region of the recognized technician 9, e.g., the position of the shoulder joint on the side where the technician 9 does not hold the ultrasound probe 4.

That is, the sensor 6 uses, for example, the distance measurement light source and the photoreceiver of the camera 61 to irradiate an examination space with infrared light and receive the reflected light of the irradiated light on the technician 9. The sensor 6 then calculates the distance L between the sensor 6 and the position of the shoulder joint of the technician 9 on the side where he/she does not hold the ultrasound probe 4. Note that since the sensor 6 is integrally attached to the upper portion of the monitor 3, the distance L can be regarded as the distance between the technician and the monitor 3.

### (b) Detection of Body Axis Angle θ of Technician 9 Relative to Vertical Direction

The angle θ of a specific region of the recognized technician 9, e.g., the body axis, relative to the vertical direction is detected in the following manner.

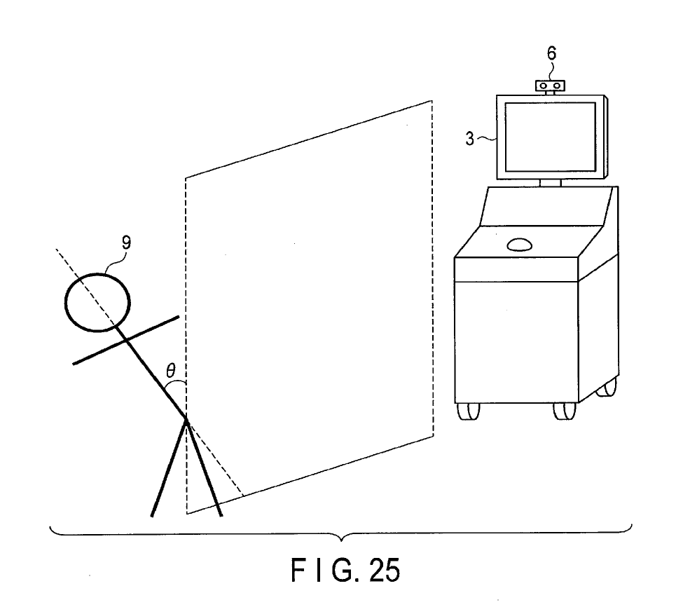

That is, the sensor 6 acquires the image of the technician 9 imaged by, for example, the camera 61. The angle θ of the body axis relative to the vertical direction is calculated based on the posture of the technician 9 on the image (see, for example, FIG. 25).

(c) Detection of (Contact/Non-Contact of Ultrasound Probe 4 Operated by Technician 9 with Respect to Object 8)

The sensor 6 acquires the image of the ultrasound probe 4 and the object 8 imaged by, for example, the camera 61. The sensor 6 then detects the contact/non-contact of the ultrasound probe 4 with respect to the object 8 based on the positional relationship between the ultrasound probe 4 and the object 8 depicted on the image.

In addition, the first modification exemplifies, instead of the conditions determined by the input acceptance condition determination program 324 in FIG. 22, the gesture/speech input acceptance conditions which are satisfied when all the following conditions are satisfied: for example, the ultrasound probe 4 being in use; the ultrasound probe being in contact with the object; and the distance between the technician 9 and the monitor 3 being equal to or more than 50 cm. In addition, the gesture/speech input acceptance conditions are satisfied when all the following conditions are satisfied: the ultrasound probe 4 being in use; the ultrasound probe being in contact with the object; and the body axis angle θ of the technician 9 being equal to or more than 30°.

As described above, under the situation like the first modification, it is possible to limit a gesture/speech input acceptance period to only a period under a situation truly required by the operator.

### Second Modification

As an example in which a technician needs to operate an ultrasound probe in a limited posture, the second modification will exemplify a case in which the toe is examined. A situation in which the toe is examined includes, for example, the case shown in FIG. 3 described in the first embodiment. The operator (technician) 7 needs to bend his/her body and bring the ultrasound probe 4 into contact with the toe of the object 8 to examine the toe. In such a case, the apparatus detects the unnatural posture of the operator 7 by detecting the body axis angle θ of the operator 7 who has bent his/her body, and performs control while using such detection as one of gesture/speech input acceptance conditions.

Detection items in the second modification which are used as gesture/speech input acceptance conditions are the same as those in the first modification. In addition, the gesture/speech input acceptance conditions are the same as those described in, for example, the first modification.

As described above, in a situation like the second modification, it is possible to further limit a gesture/speech input acceptance period to only a period under a situation truly required by the operator.

### Other Embodiments

The fourth embodiment is configured to control the direction of the screen of the monitor 3. However, if the ultrasound diagnostic apparatus is provided with an automatic traveling function, the direction of the ultrasound diagnostic apparatus itself may be changed.

In addition, the tracking function for the direction of the monitor screen described in the fourth embodiment may be added to each of the first to third embodiments.

In addition, in the fifth embodiment, the technician 9 and the surgeon 10 use only the monitor 3 and the sensor 6 of the ultrasound diagnostic apparatus. However, for example, in addition to the monitor 3 and the sensor 6 of the ultrasound diagnostic apparatus, a monitor and a sensor which are exclusively used by the surgeon 10 may be further installed and controlled.

Furthermore, the fifth embodiment is configured to perform control to accept gesture/speech input operation information by the technician 9 and the surgeon 10. However, it is also possible to perform control to accept gesture/speech input operation information from persons other than the technician 9 and the surgeon 10. Moreover, it is possible to perform control upon setting an upper limit to the number of persons from whom gesture/speech input operation information can be accepted.

In addition, the process for detecting the direction of a face, the distance detection process, the setting contents of gesture/speech input acceptance conditions, the setting contents of tracking conditions, and the like can be variously modified and implemented.

The word “probe operating state” used in the above description means a state in which an operator is operating an ultrasound probe.

The word “processor” used in the above description means circuitry such as a CPU (Central Processing Unit), a GPU (Graphics Processing Unit), an ASIC (Application Specific Integrated Circuit), a programmable logic device (e.g., an SPLD (Simple Programmable Logic Device), a CPLD (Complex Programmable Logic Device), or an FPGA (Field Programmable Gate Array)), or the like. The processor implements functions by reading out programs stored in the storage circuit and executing the programs. Note that it is possible to directly incorporate programs in the circuit of the processor instead of storing the programs in the storage circuit. In this case, the processor implements functions by reading out programs incorporated in the circuit and executing the programs. Note that each processor in each embodiment described above may be formed as one processor by combining a plurality of independent circuits to implement functions as well as being formed as a single circuit for each processor. In addition, a plurality of constituent elements in each embodiment described above may be integrated into one processor to implement its function.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. An ultrasound diagnostic apparatus comprising:
an ultrasound probe used for ultrasound transmission/reception; and
circuitry configured to:
detect a probe operating state;
determine whether the probe operating state matches a predetermined condition; and
accept an operation information input by at least one of gesture and speech by an operator of the ultrasound probe based on a determination result obtained by the determination.

2. The ultrasound diagnostic apparatus of claim 1, wherein the circuitry is configured to further detect contact/non-contact of the ultrasound probe with respect to an object as the probe operating state.

3. The ultrasound diagnostic apparatus of claim 1, wherein the circuitry is configured to:
detect a position of the operator of the ultrasound probe as the probe operating state; and
determine whether the position of the operator falls within a preset range.

4. The ultrasound diagnostic apparatus of claim 1, wherein the circuitry is configured to cause a display to display a guidance message or an icon which indicates that acceptance by at least one of gesture and speech is ready.

5. The ultrasound diagnostic apparatus of claim 1, wherein the circuitry is configured to:
detect a direction of the face of the operator based on image data obtained by imaging the operator as the probe operating state; and
determine whether the direction of the face of the operator is a direction of the display.

6. The ultrasound diagnostic apparatus of claim 1, wherein the circuitry is configured to:
detect a body axis angle of the operator with respect to a vertical direction based on the image data obtained by imaging the operator as the probe operating state; and
determine whether a body axis angle of the operator relative to a vertical direction falls within a preset range.

7. The ultrasound diagnostic apparatus of claim 1, wherein the circuitry is configured to further detect the probe operating state including a use state of the ultrasound probe, the position of the operator of the ultrasound probe, and a contact state of the ultrasound probe with respect to the object.

8. The ultrasound diagnostic apparatus of claim 1, wherein the circuitry is configured to further detect the probe operating state including a use state of the ultrasound probe, a body axis angle of the operator of the ultrasound probe with respect to a vertical direction, and a contact state of the ultrasound probe with respect to the object.

9. The ultrasound diagnostic apparatus of claim 1, wherein the circuitry is configured to further detect the probe operating state including a use state of the ultrasound probe, a direction of the face of the operator, and a position of a hand of the operator.

10. The ultrasound diagnostic apparatus of claim 1, wherein the circuitry is configured to accept an operation information input by at least one of gesture and speech from a person other than the operator upon detecting predetermined input information by at least one of gesture and speech from a person other than the operator.

11. An ultrasound diagnostic apparatus control method comprising:
detecting a probe operating state of an ultrasound probe used for ultrasound transmission/reception,
determining whether the probe operating state matches a predetermined condition, and
accepting an operation information input by at least one of gesture and speech by an operator of the ultrasound probe based on a determination result obtained by the determination.

12. The ultrasound diagnostic apparatus control method of claim 11, further comprising detecting contact/non-contact of the ultrasound probe with respect to an object as the probe operating state.

13. The ultrasound diagnostic apparatus control method of claim 11, further comprising:
detecting a position of the operator of the ultrasound probe as the probe operating state; and
determining whether the position of the operator falls within a preset range.

14. The ultrasound diagnostic apparatus control method of claim 11, further comprising causing a display to display a guidance message or an icon which indicates that acceptance by at least one of gesture and speech is ready.

15. The ultrasound diagnostic apparatus control method of claim 11, further comprising:
detecting a direction of the face of the operator based on image data obtained by imaging the operator as the probe operating state; and
determining whether the direction of the face of the operator is a direction of the display.

16. The ultrasound diagnostic apparatus control method of claim 11, further comprising:
detecting a body axis angle of the operator with respect to a vertical direction based on the image data obtained by imaging the operator as the probe operating state; and
determining whether a body axis angle of the operator relative to a vertical direction falls within a preset range.

17. The ultrasound diagnostic apparatus control method of claim 11, further comprising detecting the probe operating state including a use state of the ultrasound probe, the position of the operator of the ultrasound probe, and a contact state of the ultrasound probe with respect to the object.

18. The ultrasound diagnostic apparatus control method of claim 11, further comprising detecting the probe operating state including a use state of the ultrasound probe, a body axis angle of the operator of the ultrasound probe with respect to a vertical direction, and a contact state of the ultrasound probe with respect to the object.

19. The ultrasound diagnostic apparatus control method of claim 11, further comprising detecting the probe operating state including a use state of the ultrasound probe, a direction of the face of the operator, and a position of a hand of the operator.

20. The ultrasound diagnostic apparatus control method of claim 11, further comprising accepting an operation information input by at least one of gesture and speech from a person other than the operator upon detecting predetermined input information by at least one of gesture and speech from a person other than the operator.
